(19) United States
(12) Patent Application Publication
(10) Pub. No.: US 2023/0173047 A1
(43) Pub. Date: Jun. 8, 2023

(54) CITRULLINATED NUCLEOPHOSMIN PEPTIDES AS CANCER VACCINES

(71) Applicant: SCANCELL LIMITED, Oxford Oxfordshire (GB)

(72) Inventors: Ruhul Choudhury, Oxford Oxfordshire (GB); Linda Gillian Durrant, Oxford Oxfordshire (GB)

(21) Appl. No.: 17/920,180

(22) PCT Filed: Apr. 20, 2021
(86) PCT No.: PCT/EP2021/060175

§ 371 Date: Oct. 20, 2022

(30) Foreign Application Priority Data
Apr. 21, 2020 (GB) 2005779.0

(51) Int. Cl. A61K 39/00; A61P 35/00; C07K 14/47

(52) U.S. Cl. CPC A61K 39/0011; A61P 35/00; C07K 14/4748; A61K 2039/572; A61K 2039/55561; A61K 2039/55572

(57) ABSTRACT

The present invention relates to modified nucleophosmin peptides that can be used in cancer immunotherapy. The modified peptides may be used as vaccines or as targets for T cell receptor (TCR) and adoptive T cell transfer therapies. Such vaccines or targets may be used in the treatment of cancer.

The present invention relates to modified nucleophosmin peptides that can be used in cancer immunotherapy. The modified peptides may be used as vaccines or as targets for T cell receptor (TCR) and adoptive T cell transfer therapies. Such vaccines or targets may be used in the treatment of cancer.

In order to be effective, cancer vaccines need to induce a potent immune response that is able to break the tolerance and overcome the immunosuppressive tumour environment. The importance of CD4 T cells in mediating tumour destruction has been recently highlighted, however, the induction of self-specific CD4 responses has proved more difficult. In contrast, CD4 T cells recognising modified self-epitopes have been shown to play a role in the pathophysiology of several autoimmune diseases such as rheumatoid arthritis (RA), collagen II-induced arthritis, sarcoidosis, celiac disease and psoriasis (Choy 2012; Grunewald and Eklund 2007; Coimbra et al. 2012; Holmdahl et al. 1985). One of these common modifications is the citrullination of arginine, which involves the conversion of the positively charge aldimine group (=NH) of arginine to the neutrally charged ketone group (=O) of citrulline. Citrullination is mediated by Peptidylarginine deiminases (PADs), which are a family of calcium dependent enzymes found in a variety of tissues. A recent report by Ireland et al. (Ireland and Unanue 2011), demonstrated that the presentation of citrullinated T cell epitopes on antigen presenting cells (APCs) is also dependent upon autophagy and PAD activity. This process has also been demonstrated to be an efficient mechanism to enable processing of endogenous antigens for presentation on MHC class II molecules on professional APCs as well as epithelial cells (Munz 2012; Schmid, Pypaert, and Munz 2007). Autophagy is constitutive in APCs, but in other cells it is only induced by stress (Green and Levine 2014). T cells recognising citrullinated epitopes do not target normal healthy cells that do not express citrullinated peptides. Autophagy is triggered by stress such as hypoxia and nutrient starvation and is upregulated to promote tumour survival (Green and Levine 2014).

Nucleophosmin (NPM), also known as nucleolar phosphoprotein B23, No38, or numatrin, was first identified as a nucleolar phosphoprotein expressed at high levels in the granular regions of the nucleolus (Kang, Olson, and Busch 1974; Kang et al. 1975; Grisendi et al. 2006). NPM is ubiquitously expressed and plays a role in the regulation of cell growth, proliferation and transformation (Feuerstein, Chan, and Mond 1988); its expression rapidly increases in response to mitogenic stimuli, increased amounts of the protein can be detected in highly proliferating and malignant cells (Chan et al. 1989). NPM is a multi-functional protein that is involved in many cellular activities and has been related to both proliferative and growth-suppressive roles in the cell. Much of the interest in the NPM1 gene (Liu and Chan 1993) is due to it being implicated in human tumourigenesis. The overexpression of NPM correlates with uncontrolled cell growth and cellular transformation, whereas the disruption of NPM expression can cause genomic instability and centrosome amplification, which increases the risk of cellular transformation. NPM is frequently overexpressed in solid tumours of diverse histological origin (Tanaka et al. 1992; Nozawa et al. 1996; Shields et al. 1997; Subong et al. 1999; Tsui et al. 2004; Skaar et al. 1998; Bernard et al. 2003), whilst the NPM1 locus is involved in chromosomal translocations or deleted in various kinds of haematological malignancies and solid tumours (Redner et al. 1996; Morris et al. 1994; Yoneda-Kato et al. 1996; Falini et al. 2005; Mendes-da-Silva et al. 2000). NPM1 is one of the most frequently mutated genes in acute myeloid leukaemia (AML), having been found to be mutated and aberrantly localised in the cytoplasm of leukaemic blasts in around 35% of patients.

NPM is a highly conserved, ubiquitously expressed phosphoprotein of around 35 kDa which is mainly localised in the nucleoli, but is able to shuttle between the nucleus and cytoplasm (Borer et al. 1989; Yun et al. 2003). The shuttling activity of NPM and its proper subcellular localisation may be critical for cellular homeostasis. By shuttling between cellular compartments, NPM engages in various cellular processes, including the transport of pre-ribosomal particles, ribosome biogenesis, assisting in the transport of small basic proteins to the nucleolus, response to stress stimuli (UV irradiation and hypoxia), maintenance of genomic stability and the regulation of DNA transcription. It is regulated through SUMOylation (small ubiquitin-like modifier; by SENP3 and SENP5) is another facet of the proteins regulation and cellular functions. NPM is also involved in regulating the activity and stability of crucial tumour suppressors such as p53 and ARF.

NPM is associated with nucleolar ribonucleoprotein structures and can bind single-stranded and double-stranded nucleic acids, but preferentially binds G-quadruplex forming nucleic acids (secondary structures formed in nucleic acids by sequences that are guanine rich). NPM can also function as a molecular chaperone for proteins and nucleic acids (Hingorani, Szebeni, and Olson 2000; Szebeni and Olson 1999; Okuwaki et al. 2001). NPM belongs to a nuclear chaperone family of proteins known as nucleophosmins (Np), which all share a conserved N-terminal region. In vitro experiments using various protein substrates have shown that NPM is active in preventing the aggregation of proteins in the cellular environment (Szebeni and Olson 1999), and that it functions as a histone chaperone that is capable of histone assembly, nucleosome assembly and increasing acetylation-dependent transcription (Okuwaki et al. 2001; Swaminathan et al. 2005).

The most prevalent form of NPM is NPM1.1 (also called B23.1). Two alternatively spliced isoforms, designated NPM1.3 (also called B23.2) and NPM1.2 also exist No function has been found for NPM1.2. NPM1.1 is the most prevalent form in all tissues (Chang and Olson 1990; Wang, Umekawa, and Olson 1993). Under native conditions, NPM exists as an oligomer (Herrera et al. 1996), but can also form pentamers and decamers (Namboodiri et al. 2004).

The diversity of the cellular activities that NPM is involved in make it both a potential oncogene and a potential tumour suppressor. The deregulation of NPM expression and/or localisation could through different mechanisms contribute to tumourigenesis. The NPM protein is overexpressed in various tumours, and has been proposed as a marker for gastric (Tanaka et al. 1992), colon (Nozawa et al. 1996), ovarian (Shields et al. 1997) and prostate (Subong et al. 1999) carcinomas. In some cases, the expression levels of NPM have been correlated with the stage of tumour progression. For example, overexpression of NPM mRNA is independently associated with the recurrence of bladder carcinoma and progression to a more advanced stage of disease (Tsui et al. 2004). Proteomic analysis identified NPM as an oestrogen-regulated protein that is associated with acquired oestrogen-independence in human breast cancer cells (Skaar et al. 1998), suggesting the status of NPM expression can be correlated to specific pathophysiological features.

NPM is able to bind to many partners in distinct cellular compartments, including nucleolar factors, transcription factors, histones, proteins involved in cell proliferation, and the response to oncogenic stress. Post-translational modifications of proteins occur under conditions of cellular stress, one such modification involves citrullination, the conversion of arginine residues to citrulline by peptidylarginine deiminase (PAD) enzymes. Citrullination occurs as a result of a degradation and recycling process (autophagy) that is induced in stressed cells (Ireland and Unanue 2011). Citrullinated epitopes can subsequently be presented on MHC class II molecules for recognition by CD4 T cells. The potent immune responses unleashed in response to citrullinated proteins can be harnessed and redirected to destroy cancer cells. This immune response is mediated by killer CD4 T cells that then secrete high amounts of IFNy. This increased MHC class II expression and then directly kills the tumour cells, without the need for CD8 T cell involvement (Brentville et al. 2016; Durrant, Metheringham, and Brentville 2016). Tumour recognition depends upon both citrullination and autophagy. A high number of IFNy secreting CD4 T cells have been shown to be induced following immunisation of mice with two citrullinated peptides derived from the cytoskeletal protein, vimentin. Ex-vivo, these CD4 T cells recognise tumour cells in which autophagy is induced by either starvation or rapamycin. Vimentin’s function and expression in tumours has been detailed previously in WO2014023957.

Much interest in NPM has arisen since the discovery of heterozygous mutations in the terminal exon of the NPM1 gene. The NPM1 gene maps to chromosome 5q35 and is expressed in three isoforms though alternative splicing. NPM1.1 (P06748-1) (294 residues) is the most abundant one and displays nucleolar localisation. Isoform NPM1.2 (P06748-2) lacks an in frame exon (exon 8) resulting in a shorter protein with respect to NPM1.1 in which an internal segment (residues 195-223) is lacking. NPM1.3 (P06748-3) uses an alternative exon at the 3′ end, which is responsible for a shorter protein construct lacking the last 35 amino acids with respect to NPM1.1 (Wang, Umekawa, and Olson 1993); this isoform is expressed at low levels and has a nucleoplasmic localisation. The most abundant NPM1.1 isoform (NPM1) is expressed in all tissues.

The NPM1 gene is up-regulated, mutated and chromosomally translocated in many tumour types; chromosomal aberrations have been found in patients with non-Hodgkin lymphoma, acute promyelocytic leukaemia, myelodysplastic syndrome, and acute myelogenous leukaemia (Falini et al. 2007). Majority of NPM mutations ultimately affects the tryptophan residue at 288 or 290 amino acid position of the 294 amino acid NPM protein (Duployez et al. 2018). More than half of the mutations are also associated with normal karyotype (Duployez et al. 2018). However, in anaplastic large cell lymphoma (ALCL) which represents 2-8% of adult non-Hodgkin lymphoma and in small cases of AML NPM gene is translocated with other genes (Jairajpuri et al. 2014). In ACL, ALK on chromosome 2 is fused with NPM on chromosome 5 whereas in <1% of cases of AML translocation generates a chimeric gene named NPM MLF1 (myelodysplasia/myeloid leukemia factor 1) due to a fusion between chromosome 3 and 5 (Falini et al. 2007). Extremely rare translocation in acute promyelocytic leukemia (APL) affecting only three cases so far involves fusion of NPM1 gene with the retinoic acid receptor-a gene (RAR_{A}) (Falini et al. 2007). Heterozygous mice for NPM1 are vulnerable to tumour development. NPM is also frequently overexpressed in a variety of solid tumours of different histological origin (prostate (Leotoing et al. 2008), liver (Yun et al. 2007), thyroid (Pianta et al. 2010), colon (Nozawa et al. 1996), gastric (Tanaka et al. 1992), pancreas (Zhu et al. 2015), glioma and glioblastoma (Chen et al. 2015; Holmberg Olausson et al. 2015), astrocytoma (Kuo et al. 2015) and others) and, in many cases, its overexpression correlates with mitotic index and metastasis. Thus, as NPM is expressed by a range of solid and haematological cancers, it is a highly attractive target for a vaccine. Further, specific antibodies to mutated versions exist, allowing for diagnostic assays to be performed to identify patients who have the potential to benefit from NPM targeted therapy.

NPM has been shown to interact with AKT1 (Lee et al. 2008), BARD1 (Sato et al. 2004), BRCA1 (Sato et al. 2004) and nucleolin (Li et al. 1996) and has multiple binding partners (Lindstrom 2011). It is thought NPM1 can promote tumour growth by the inactivation of the tumour suppressor p53/ARF pathway although when expressed at low levels, NPM1 can suppress tumour growth by the inhibition of centrosome duplication.

NPM can be translocated to the nucleoplasm during periods of serum starvation or treatment with anticancer drugs, where is phosphorylated. NPM is a promising target for the treatment of both haematologic and solid malignancies. Over the past decade several molecules that target NPM1 have been discovered and their effect and therapeutic potential investigated. A striking synergy has been observed in many cases when NPM targeting compounds were administered in combination with different chemotherapeutic agents or radiotherapy, this suggests that interfering with NPM may sensitise cancer cells. NSC348884, Rev37-47, 1A1 RNA aptamer, CIGB-300, avrainvillamide, all-trans-retinoic-acid (ATRA), YTR107 and NucAnt 6L (N6L) are the NPM binding compounds that have been tested in vitro or in clinic (Di Matteo et al. 2016). Some compounds such as NSC348884 and 1A1 RNA aptamer prevent oligomerisation of NPM therefore making the protein unstable (Qi et al. 2008; Jian et al. 2009), while direct binding of Rev37-47 and avrainvillamide to NPM impedes its function (Wulff, Siegrist, and Myers 2007; Szebeni, Herrera, and Olson 1995). ATRA is one of the main treatment options for APL (Lo-Coco et al. 2013) and in vitro study suggested mutated form of NPM undergo proteasomal degradation following binding to ATRA (Martelli et al. 2015; Di Matteo et al. 2016). YTR107 can induce radiosensitisation of cancer cells through NPM by interfering with DNA repair mechanism (Di Matteo et al. 2016; Sekhar et al. 2014). Although mechanistic pathways are different the net result of these compounds binding to NPM was apoptosis of cancer cells in vitro. Two compounds (CIGB-300, NPM phosphorylation inhibitor and Nucant N6L (rich in lysine and arginine residues)) have entered clinical trials to date. The detailed mechanism of cancer cells apoptosis via targeting NPM is still to be elucidated however, evidence so far suggests compounds targeting NPM are a promising target for cancer treatment.

Under cellular stress and DNA damage, P53 gene is activated. Tanikawa et al. showed P53 induced transactivation of PAD4 leads to NPM citrullination (Tanikawa et al. 2009). Both PAD4 and NPM are generally nucleus based, transfection of HEK293T cells with PAD4 resulted in localisation of NPM from nucleoli to nucleoplasm indicating PAD4 mediated NPM citrullination takes place in the nucleus (Tanikawa et al. 2009). In parallel, NPM was also immunoprecipitated from whole cells lysates and mass spectrometry performed. A B cell epitope containing citrulline at position 197 was identified. This was then used to generate an antibody (Tanikawa et al. 2009). Our results demonstrate the peptide containing citrulline at residue 197 failed to induce a T cell response, hence confirming that this is a B cell epitope. In contrast, in this invention NPM peptides comprising novel citrullinated position show strong T cell responses resulting in anti-tumour immunity.

According to a first aspect of the invention, there is provided a citrullinated T cell antigen comprising, consisting essentially of or consisting of,
(i) the amino acid sequence AKFINYVKNCFRMTD wherein the arginine (R) residue is replaced with citrulline, or
(ii) the amino acid sequence of i), with the exception of 1, 2 or 3 amino acid substitutions, and/or 1, 2 or 3 amino acid insertions, and/or 1, 2 or 3 amino acid deletions in a non-arginine position.

The inventors have unexpectedly found that it is possible to raise T cell responses to certain antigens from NPM expressed on tumour cells in which the arginine has been replaced by citrulline. Furthermore, citrulline-containing peptides permit the development of T cell-based therapies, including but not limited to tumour vaccines, as well as T cell receptor (TCR) and adoptive T cell transfer therapies.

The T cell antigen of the present invention may be a MHC class II antigen, i.e. form a complex with and be presented on a MHC class II molecule. The skilled person can determine whether or not a given polypeptide forms a complex with an MHC molecule by determining whether the MHC can be refolded in the presence of the polypeptide. If the polypeptide does not form a complex with MHC, the MHC will not refold properly. Refolding is commonly confirmed using an antibody that recognises MHC in a folded state only. Further details can be found in (Garboczi, Hung, and Wiley 1992).

All of the arginine amino acid residues in the antigen may be converted to citrulline. Alternatively, 1, 2, 3 or 4 of the arginine amino acid residues in the antigen may be converted to citrulline, with the remainder being unconverted. Thus, an antigen of the present invention may have 1, 2, 3 or 4 citrulline residues. Antigens of the present invention may be up to 25 amino acids in length. They may be at least 5 amino acids in length and may be no longer than 18, 19, 20, 21, 22, 23 or 24 amino acids. The T cell antigen of the present invention may tumour-associated and may stimulate an immune response against the tumour.

The inventors have shown that, in normal healthy donors and HLA transgenic mice, T cells recognising citrullinated NPM peptides produce IFNy and can be detected following stimulation with NPM peptides. They have also shown that certain citrullinated NPM peptides generate a T cell response in vivo and, as such, can be used as a vaccine target for cancer therapy. The inventors have shown that the anti-tumour response was lost in B16F1cDP4PAD2KO tumour bearing mice. This demonstrates that PAD2 is critical for the citrullination of arginine 277 in the tumour cells in vivo and for the anti-tumour effects. These results show that PAD4 citrullinates different residues within the nucleus for regulation of expression of NPM but PAD2, which is predominantly expressed with the cytoplasm, is responsible for citrullination of residues of NPM for MHC-II presentation.

The T cell antigen of the present invention may comprise, consist essentially of, or consist of i) one or more of the following amino acid sequences wherein the arginine (R) residue is replaced with citrulline:

AKFINYVKNCFRMTDQEAIQ
LPKVEAKFINYVKNCFRMTD, or

ii) one or more of the amino acid sequences of i), with the exception of 1, 2 or 3 amino acid substitutions, and/or 1, 2 or 3 amino acid insertions, and/or 1, 2 or 3 amino acid deletions in a non-arginine position. The antigen may have a total of 1, 2, 3, 4 or 5 amino acid modifications selected from substitutions, insertions and substitutions in a non-arginine position. The T cell antigen of ii) is preferably capable of raising an immune response against tumours including, but not restricted to, Acute Myeloid Leukaemia (AML), lung, colorectal, renal, breast, ovary and liver tumours.

It is preferred if the T cell antigen of the present invention comprises, consists essentially of, or consists of i) one or more of the following amino acid sequences:

AKFINYVKNCF-cit-MTD (NPM 266-280)
AKFINYVKNCFcitMTDQEAIQ (NPM 266-285)
LPKVEAKFINYVKNCFcitMTD (NPM 261-280)

wherein “cit” represents citrulline, or ii) the amino acid sequence of i), with the exception of 1, 2 or 3 amino acid substitutions, and/or 1, 2 or 3 amino acid insertions, and/or 1, 2 or 3 amino acid deletions in a non-citrulline position.

The inventors have unexpectedly found that certain citrullinated peptides derived from NPM can be used to raise an immune response against tumours including, but not restricted to, AML, lung, colorectal, renal, breast, ovary and liver tumours. The inventors have shown that LPKVEAKFINYVKNCFcitMTD - NPM 261-280 citrullinated at position 277

AKFINYVKNCFcitMTDQEAIQ - NPM 266-285 citrullinated at position 277 generated an anti-tumour immune response in vivo to citrullinated NPM epitope. These two peptides are homologous to mouse and therefore are not recognised as foreign.

Citrullinated peptides are known to stimulate T cell responses in autoimmune patients with the shared HLA-DR4 motif. In contrast, the inventors are the first to show that citrullinated NPM peptides, such as NPM 266-285 citrullinated at position 277 and NPM 261-280 citrullinated at position 277, can stimulate potent T cell responses in HLA-DP4 and DR4 transgenic mice. All healthy donors showing responses to NPM 266-285 citrullinated at position 277 expressed HLA-DP4. In addition, two of the donors also expressed HLA-DR4. This makes it a promising vaccine for the treatment of haematological and solid tumours in a wider population. The response to NPM 266-285 citrullinated at position 277 was the strongest and showed minimal reactivity to the unmodified wildtype sequence. T cells recognising this citrullinated peptide antigen can target tumour cells and elicit strong anti-tumour effects in vivo, thus providing the first evidence for the use of citrullinated NPM 266-285 cit as a vaccine target for cancer therapy. There was a strong anti-tumour response with NPM 261-280 citrullinated at position 277 against tumours which expressed MHC-II but a much weaker response against tumours that could not express MHC-II. This suggests that in MHC-II negative tumours CD4 T cells can induce anti-tumour responses by bystander effects on CD8 and/or NK responses but the superior anti-tumour responses when tumour express MHC-II suggests that the CD4 T cells can also mediate direct tumour killing.

The MHC class II antigen processing pathway can be influenced by many factors, such as the internalisation and processing of exogenous antigen, the peptide binding motif for each MHC class II molecule and the transportation and stability of MHC class II: peptide complex. The MHC class II peptide binding groove is open at both ends and it is less constrained by the length of the peptide compared to MHC Class I molecules. The peptides that bind to MHC class II molecules range in length from 13-25 amino acids long and typically protrude out of the MHC molecule (Kim et al. 2014; Sette et al. 1989). These peptides contain a consecutive stretch of nine amino acids, referred to as the core region. Some of these amino acids interact directly with the peptide binding groove (Andreatta et al. 2017). The amino acids either side of the core peptide protrude out of the peptide binding groove; these are known as peptide flanking regions. They can also impact peptide binding and subsequent interactions with T cells (Arnold et al. 2002; Carson et al. 1997; Godkin et al. 2001). The length of MHC class II peptides allows long peptides, e.g. 15-20 mers, to be used in screening. For example, in NPM, this would require 71 × 15 mer overlapping peptides; these cover the full 294 amino acids and overlap by 11 amino acids. (Alternatively, if 20 mers overlapping by 15 were used, it would require 56 × 20 mer peptides). Of these 71 peptides, 28 contain an Arginine residue. To screen 28 peptides, these can be combined into smaller peptide pools and incorporated into an in vitro assay or used in in vivo murine immunisation studies. This type of screening is standard in designing neoepitope personalised vaccines to screen hundreds of peptides. For example, Liu et al. examined responses to neoantigens in epithelial ovarian cancer patients (Liu et al. 2019). They screened 75 peptides and found 27 that stimulated T cell responses. Bobisse et al. screened 776 peptides and found 15 (2%) that stimulated T cell responses (Bobisse et al. 2018).

This method is also a viable approach to identify MHC class I peptides as longer 20 mer peptides also can contain nested MHCI restricted epitopes and has been used to identify both MHC class II and MHC class I restricted CD4 and CD8 T cell responses. Given the use of such methodology for identifying cancer vaccine neoantigen targets for individual cancer patients, it is an equally viable and justifiable approach for single antigens in order to develop a vaccine to treat a wide range of cancer patients whose tumour expresses the citrullinated antigen. This would require testing in multiple donors to ensure epitopes binding to different MHC class II and MHC class I molecules are identified. The same 71 × 15 mer or 56 × 20 mer overlapping peptides would be used either individually or in pools in each donor.

MHC class II molecules are highly polymorphic, the peptide binding motifs are highly degenerate with many promiscuous peptides having been identified that can bind multiple MHC class II molecules (Consogno et al. 2003). The amino acids that are critical for peptide binding have been identified from crystallography studies of MHC class II:peptide complexes (Corper et al. 2000; Dessen et al. 1997; Fremont et al. 1996; Ghosh et al. 1995; Latek et al. 2000; Li et al. 2000; Lee, Wucherpfennig, and Wiley 2001; Brown et al. 1993; Smith et al. 1998; Stern et al. 1994; Scott et al. 1998; Fremont et al. 1998). These studies have indicated that P1, P4, P6 and P9 always point towards the MHC whereas P-1, P2, P5 P8 and P11 always orient towards the TCR. The frequency of HLA-DR and HLA-DP alleles is listed in Table 1 (Thomsen and Nielsen 2012; Gonzalez-Galarza et al. 2015).

TABLE 1

HLA-DR and DP allele frequency in the UK population

| Allele | % individuals that have the allele | Sample Size |
|---|---|---|
| DRB1*04 | 32 | 57,732 |
| DRB1*03 | 28 | 57,732 |
| DRB1*07 | 28 | 57,732 |
| DRB1*15 | 28 | 57,732 |
| DBR1*01 | 22 | 57,732 |
| DPB1*0401 | 70 | 187 |
| DPB1*0301 | 22 | 187 |
| DPB1*0201 | 20 | 187 |
| DPB1*0101 | 14 | 187 |

In contrast, MHC class I molecules show more restricted peptide binding properties. Amino acids critical for binding to MHC class I have also been identified through prediction algorithms analysing known naturally binding peptides (Jurtz et al. 2017), which indicated that (with the exception of HLA-B*0801) P2 and P9 orient towards the MHC acting as binding anchor residues.

The most prevalent form of Nucleophosmin is NPM1.1. Two alternatively spliced isoforms, designated NPM1.3 and NPM1.2, also exist, with NPM1.1 being the prevalent form in all tissues. The peptides LPKVEAKFINYVKNCFRMTD (261-280) and AKFINYVKNCFRMTDQEAIQ (266-285) are only found in NPM1.1 (B23.1) and NPM1.3 (B23.2). Accordingly, NPM 261-280 and 266-285 citrullinated at position 277, as well as nucleic acids encoding it, can be used for targeting the most prevalent form of NPM (NPM1.1). In NPM1.3 the corresponding peptides are located at 232-251 and 237-256 citrullinated at position 248.

NPM is highly conserved between those species in which the gene has been cloned (chicken, mouse, dog, sheep, cow, horse, pig and human). Accordingly, an antigen of the invention, optionally in combination with a nucleic acid comprising a sequence that encodes such an antigen, can be used for treating cancer in non-human mammals.

The invention also includes within its scope peptides having the amino acid sequence as set out above and sequences having substantial identity thereto, for example, at least 70%, at least 80%, at least 85%, at least 90%, at least 95% or at least 99% identity thereto, as well as their use in medicine and in particular in a method for treating cancer. Such peptides are preferably capable of raising an immune response against tumours including, but not restricted to, AML, lung, colorectal, renal, breast, ovary and liver tumours. The percent identity of two amino acid sequences or of two nucleic acid sequences is generally determined by aligning the sequences for optimal comparison purposes (e.g., gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e. % identity = number of identical positions/total number of positions x 100).

The determination of percent identity between two sequences can be accomplished using a mathematical algorithm known to those of skill in the art. An example of a mathematical algorithm for comparing two sequences is the algorithm of Karlin and Altschul (Karlin and Altschul 1993). The NBLAST and XBLAST programs of Altschul, et al. have incorporated such an algorithm (Altschul et al. 1990). BLAST nucleotide searches can be performed with the NBLAST program (score = 100, word length = 12) to obtain nucleotide sequences homologous to a nucleic acid molecules of the invention. BLAST protein searches can be performed with the XBLAST program (score = 50, word length = 3) to obtain amino acid sequences homologous to a protein molecules of the invention. To obtain gapped alignments for comparison purposes, Gapped BLAST can be utilized as described in (Altschul et al. 1997). Alternatively, PSI-Blast can be used to perform an iterated search that detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller (Myers and Miller 1989). The ALIGN program (version 2.0) which is part of the GCG sequence alignment software package has incorporated such an algorithm. Other algorithms for sequence analysis known in the art include ADVANCE and ADAM as described in (Torelli and Robotti 1994) and FASTA described in (Pearson and Lipman 1988). Within FASTA, ktup is a control option that sets the sensitivity and speed of the search.

Amino acid substitution means that an amino acid residue is substituted for a replacement amino acid residue at the same position. Inserted amino acid residues may be inserted at any position and may be inserted such that some or all of the inserted amino acid residues are immediately adjacent one another or may be inserted such that none of the inserted amino acid residues is immediately adjacent another inserted amino acid residue.

The antigen of the invention may comprise one, two or three additional amino acids at the C-terminal end and/or at the N-terminal end thereof. An antigen of the invention may comprise the amino acid sequence set out above with the exception of one amino acid substitution and one amino acid insertion, one amino acid substitution and one amino acid deletion, or one amino acid insertion and one amino acid deletion. An antigen of the invention may comprise the amino acid sequence set out above, with the exception of one amino acid substitution, one amino acid insertion and one amino acid deletion.

Inserted amino acids and replacement amino acids may be naturally occurring amino acids or may be non-naturally occurring amino acids and, for example, may contain a non-natural side chain. Such altered peptide ligands are discussed further in (Douat-Casassus et al. 2007; Hoppes et al. 2014) and references therein). If more than one amino acid residue is substituted and/or inserted, the replacement/inserted amino acid residues may be the same as each other or different from one another. Each replacement amino acid may have a different side chain to the amino acid being replaced.

Preferably, antigens of the invention bind to MHC in the peptide binding groove of the MHC molecule. Generally, the amino acid modifications described above will not impair the ability of the peptide to bind MHC. In a preferred embodiment, the amino acid modifications improve the ability of the peptide to bind MHC. For example, mutations may be made at positions which anchor the peptide to MHC. Such anchor positions and the preferred residues at these locations are known in the art.

An antigen of the invention may be used to elicit an immune response. If this is the case, it is important that the immune response is specific to the intended target in order to avoid the risk of unwanted side effects that may be associated with an “off target” immune response. Therefore, it is preferred that the amino acid sequence of a polypeptide of the invention does not match the amino acid sequence of a peptide from any other protein(s), in particular, that of another human protein. A person of skill in the art would understand how to search a database of known protein sequences to ascertain whether an antigen according to the invention is present in another protein.

Antigens of the invention can be synthesised easily by Merrifield synthesis, also known as solid phase synthesis, or any other peptide synthesis methodology. GMP grade polypeptide is produced by solid-phase synthesis techniques by Multiple Peptide Systems, San Diego, CA. Alternatively, the peptide may be recombinantly produced, if so desired, in accordance with methods known in the art. Such methods typically involve the use of a vector comprising a nucleic acid sequence encoding the polypeptide to be expressed, to express the polypeptide in vivo; for example, in bacteria, yeast, insect or mammalian cells. Alternatively, in vitro cell-free systems may be used. Such systems are known in the art and are commercially available for example from Life Technologies, Paisley, UK. The antigens may be isolated and/or may be provided in substantially pure form. For example, they may be provided in a form which is substantially free of other polypeptides or proteins. Peptides of the invention may be synthesised using Fmoc chemistry or other standard techniques known to those skilled in the art.

In a second aspect, the invention provides a complex of the antigen of the first aspect and an MHC molecule. Preferably, the antigen is bound to the peptide binding groove of the MHC molecule. The MHC molecule may be MHC class II. The MHC class II molecule may be a DP, DR or DQ allele, such as HLA-DR4, DR1, DP4, DP2, DP5, DQ2, DQ3, DQ5 and DQ6. HLA-DR4 and DP4 are preferred.

The antigen and complex of the invention may be isolated and/or in a substantially pure form. For example, the antigen and complex may be provided in a form which is substantially free of other polypeptides or proteins. It should be noted that in the context of the present invention, the term “MHC molecule” includes recombinant MHC molecules, non-naturally occurring MHC molecules and functionally equivalent fragments of MHC, including derivatives or variants thereof, provided that peptide binding is retained. For example, MHC molecules may be fused to a therapeutic moiety, attached to a solid support, in soluble form, and/or in multimeric form.

Methods to produce soluble recombinant MHC molecules with which antigens of the invention can form a complex are known in the art. Suitable methods include, but are not limited to, expression and purification from E. coli cells or insect cells. Alternatively, MHC molecules may be produced synthetically, or using cell free systems.

Antigens and/or antigen-MHC complexes of the invention may be associated with a moiety capable of eliciting a therapeutic effect. Such a moiety may be a carrier protein which is known to be immunogenic. KLH (keyhole limpet hemocyanin) is an example of a suitable carrier protein used in vaccine compositions. Alternatively, the antigens and/or antigen-MHC complexes of the invention may be associated with a fusion partner. Fusion partners may be used for detection purposes, or for attaching said antigen or MHC to a solid support, or for MHC oligomerisation. The MHC complexes may incorporate a biotinylation site to which biotin can be added, for example, using the BirA enzyme. Other suitable fusion partners include, but are not limited to, fluorescent, or luminescent labels, radiolabels, nucleic acid probes and contrast reagents, antibodies, or enzymes that produce a detectable product. Detection methods may include flow cytometry, microscopy, electrophoresis or scintillation counting.

Antigen-MHC complexes of the invention may be provided in soluble form or may be immobilised by attachment to a suitable solid support. Examples of solid supports include, but are not limited to, a bead, a membrane, sepharose, a magnetic bead, a plate, a tube, a column. Antigen-MHC complexes may be attached to an ELISA plate, a magnetic bead, or a surface plasmon resonance biosensor chip. Methods of attaching antigen-MHC complexes to a solid support are known to the skilled person, and include, for example, using an affinity binding pair, e.g. biotin and streptavidin, or antibodies and antigens. In a preferred embodiment antigen-MHC complexes are labelled with biotin and attached to streptavidin-coated surfaces.

Antigen-MHC complexes of the invention may be in multimeric form, for example, dimeric, or tetrameric, or pentameric, or octomeric, or greater. Examples of suitable methods for the production of multimeric peptide MHC complexes are described in (Greten and Schneck 2002) and references therein. In general, antigen-MHC multimers may be produced using antigen-MHC tagged with a biotin residue and complexed through fluorescent labelled streptavidin. Alternatively, multimeric antigen-MHC complexes may be formed by using immunoglobulin as a molecular scaffold. In this system, the extracellular domains of MHC molecules are fused with the constant region of an immunoglobulin heavy chain separated by a short amino acid linker. Antigen-MHC multimers have also been produced using carrier molecules such as dextran (WO02072631). Multimeric antigen-MHC complexes can be useful for improving the detection of binding moieties, such as T cell receptors, which bind said complex, because of avidity effects.

The antigens of the invention may be presented on the surface of a cell in complex with MHC. Thus, the invention also provides a cell presenting on its surface a complex of the invention. Such a cell may be a mammalian cell, preferably a cell of the immune system, and in particular a specialised antigen presenting cell such as a dendritic cell or a B cell. Other preferred cells include T2 cells (Hosken and Bevan 1990). Cells presenting the antigen or complex of the invention may be isolated, preferably in the form of a population, or provided in a substantially pure form. Said cells may not naturally present the complex of the invention, or alternatively said cells may present the complex at a level higher than they would in nature. Such cells may be obtained by pulsing said cells with the antigen of the invention. Pulsing involves incubating the cells with the antigen for several hours using polypeptide concentrations typically ranging from 10⁻⁵ to 10⁻¹² M. Cells may be produced recombinantly. Cells presenting antigen of the invention may be used to isolate T cells and T cell receptors (TCRs) which are activated by, or bind to, said cells, as described in more detail below.

Peptides of the invention may be synthesised using Fmoc chemistry or other standard techniques known to those skilled in the art.

Another convenient way of producing a peptide according to the present invention is to express the nucleic acid encoding it, by use of nucleic acid in an expression system. Such a nucleic acid forms another aspect of the invention.

The skilled person will be able to determine substitutions, deletions and/or additions to such nucleic acids which will still provide a peptide of the present invention. The nucleic acid may be DNA, cDNA, or RNA such as mRNA obtained by cloning or produced by chemical synthesis. For therapeutic use, the nucleic acid is preferably in a form capable of being expressed in the subject to be treated. The peptide of the present invention or the nucleic acid of the present invention may be provided as an isolate, in isolated and/or purified form, or free or substantially free of material with which it is naturally associated. In the case of a nucleic acid, it may be free or substantially free of nucleic acid flanking the gene in the human genome, except possibly one or more regulatory sequence(s) for expression. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding a peptide of the present invention can be readily prepared by the skilled person, for example using the information and references contained herein and techniques known in the art (for example, see (Sambrook 1989; Ausubel 1992)), given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding the polypeptide may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially-available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

The present invention also provides constructs in the form of plasmids, vectors, transcription or expression cassettes which comprise at least one nucleic acid as described above. The present invention also provides a recombinant host cell which comprises one or more constructs as above. As mentioned, a nucleic acid encoding a peptide of the invention forms an aspect of the present invention, as does a method of production of the composition which method comprises expression from encoding nucleic acid. Expression may conveniently be achieved by culturing under appropriate conditions recombinant host cells containing the nucleic acid. Following production by expression, a composition may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli. The expression of antibodies and antibody fragments in prokaryotic cells such as E. coli is well established in the art. Expression in eukaryotic cells in culture is also available to those skilled in the art as an option for production of a specific binding member, see for recent review, for example (Reff 1993; Trill, Shatzman, and Ganguly 1995). For a review, see for example (Pluckthun 1991). Expression in eukaryotic cells in culture is also available to those skilled in the art as an option for production of a specific binding member, see for recent review, for example (Reff 1993; Trill, Shatzman, and Ganguly 1995).

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Molecular Cloning: A Laboratory Manual (Sambrook 1989). Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology (Ausubel 1992).

Thus, a further aspect of the present invention provides a host cell, which may be isolated, containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene.

In one embodiment, the nucleic acid of the invention is integrated into the genome (e.g. chromosome) of the host cell. Integration may be promoted by inclusion of sequences which promote recombination with the genome, in accordance with standard techniques.

The present invention also provides a method which comprises using a construct as stated above in an expression system in order to express a polypeptide as described above.

Polypeptides of the invention can be used to identify and/or isolate binding moieties that bind specifically to the polypeptide of the invention. Such binding moieties may be used as immunotherapeutic reagents and may include antibodies. Therefore, in a further aspect, the invention provides a binding moiety that binds the polypeptide of the invention.

Antigens and complexes of the invention can be used to identify and/or isolate binding moieties that bind specifically to the antigen and/or the complex of the invention. Such binding moieties may be used as immunotherapeutic reagents and may include antibodies and TCRs.

In a third aspect, the invention provides a binding moiety that binds the antigen of the invention. Preferably the binding moiety binds the antigen when said polypeptide is in complex with MHC. In the latter instance, the binding moiety may bind partially to the MHC, provided that it also binds to the antigen. The binding moiety may bind only the antigen, and that binding may be specific. The binding moiety may bind only the antigen-MHC complex and that binding may be specific.

When used with reference to binding moieties that bind the complex of the invention, “specific” is generally used herein to refer to the situation in which the binding moiety does not show any significant binding to one or more alternative antigen-MHC complexes other than the antigen-MHC complex of the invention.

The binding moiety may be a T cell receptor (TCR). TCRs are described using the International Immunogenetics (IMGT) TCR nomenclature, and links to the IMGT public database of TCR sequences. The unique sequences defined by the IMGT nomenclature are widely known and accessible to those working in the TCR field. For example, they can be found in the “T cell Receptor Factsbook”, (2001) LeFranc and LeFranc, Academic Press, ISBN 0-12-441352-8; Lefranc, (2011), Cold Spring Harb Protoc 2011(6): 595-603; Lefranc, (2001), Curr Protoc Immunol Appendix 1: Appendix 10; (Lefranc 2003), and on the IMGT website (www.IMGT.org). Briefly, alpha beta TCRs consist of two disulphide linked chains. Each chain (alpha and beta) is generally regarded as having two domains, namely a variable and a constant domain. A short joining region connects the variable and constant domains and is typically considered part of the alpha variable region. Additionally, the beta chain usually contains a short diversity region next to the joining region, which is also typically considered part of the beta variable region.

The TCRs may be in any format known to those in the art. For example, the TCRs may be αβ heterodimers, or they may be in single chain format (such as those described in WO9918129). Single chain TCRs include αβ TCR polypeptides of the type: Vα-L-Vβ, Vβ-L-Vα, Vα-Cα-L-Vβ, Vα-L-Vβ-Cβ or Vα- Cα -L-Vβ-Cβ, optionally in the reverse orientation, wherein Vα and Vβ are TCR α and β variable regions respectively, Cα and Cβ are TCR α and β constant regions respectively, and L is a linker sequence. The TCR may be in a soluble form (i.e. having no transmembrane or cytoplasmic domains); or may contain full length alpha and beta chains. The TCR may be provided on the surface of a cell, such as a T cell. The cell may be a mammalian cell, such as a human cell.

The cell may be used in medicine, in particular for treating cancer. The cancer may be a solid tumour or a haematological neoplasia. The cancer may be lung, colorectal, renal, breast, ovary and liver cancer, acute myeloid leukaemia. The cells may be autologous to the subject to be treated or not autologous to the subject to be treated.

The alpha and/or beta chain constant domain of the TCR may be truncated relative to the native/naturally occurring TRAC/TRBC sequences. In addition, the TRAC/TRBC may contain modifications. For example, the alpha chain extracellular sequence may include a modification relative to the native/naturally occurring TRAC whereby amino acid T48 of TRAC, with reference to IMGT numbering, is replaced with C48. Likewise, the beta chain extracellular sequence may include a modification relative to the native/naturally occurring TRBC1 or TRBC2 whereby S57 of TRBC1 or TRBC2, with reference to IMGT numbering, is replaced with C57. These cysteine substitutions relative to the native alpha and beta chain extracellular sequences enable the formation of a non-native interchain disulphide bond which stabilises the refolded soluble TCR, i.e. the TCR formed by refolding extracellular alpha and beta chains (WO 03/020763). This non-native disulphide bond facilitates the display of correctly folded TCRs on phage (Li et al. 2005). In addition, the use of the stable disulphide linked soluble TCR enables more convenient assessment of binding affinity and binding half-life. Alternative positions for the formation of a non-native disulphide bond are described in WO 03/020763.

The variable domain of each chain is located N-terminally and comprises three Complementarity Determining Regions (CDRs) embedded in a framework sequence (FR). The CDRs comprise the recognition site for peptide-MHC binding. There are several genes coding for alpha chain variable (Vα) regions and several genes coding for beta chain variable (Vβ) regions, which are distinguished by their framework, CDR1 and CDR2 sequences, and by a partly defined CDR3 sequence. The Vα and Vβ genes are referred to in IMGT nomenclature by the prefix TRAV and TRBV respectively (Folch et al. 2000; Lefranc 2001) “T cell Receptor Factsbook”, Academic Press). Likewise there are several joining or J genes, termed TRAJ or TRBJ, for the alpha and beta chain respectively, and for the beta chain, a diversity or D gene termed TRBD (Folch et al. 2000; Lefranc 2001) “T cell Receptor Factsbook”, Academic Press). The huge diversity of T cell receptor chains results from combinatorial rearrangements between the various V, J and D genes, which include allelic variants, and junctional diversity (Arstila et al. 1999) (Robins et al. 2009). The constant, or C, regions of TCR alpha and beta chains are referred to as TRAC and TRBC respectively (Lefranc, (2001), Curr Protoc Immunol Appendix 1: Appendix 10). TCRs of the invention may be engineered to include mutations. Methods for producing mutated high affinity TCR variants such as phage display and site directed mutagenesis and are known to those in the art (for example see WO 04/044004 and Li et al. (Li et al. 2005)).

TCRs may also be may be labelled with an imaging compound, for example a label that is suitable for diagnostic purposes. Such labelled high affinity TCRs are useful in a method for detecting a TCR ligand selected from CD1-antigen complexes, bacterial superantigens, and MHC-peptide/superantigen complexes, which method comprises contacting the TCR ligand with a high affinity TCR (or a multimeric high affinity TCR complex) which is specific for the TCR ligand; and detecting binding to the TCR ligand. In multimeric high affinity TCR complexes such as those described in Zhu et al., (Zhu et al. 2006), (formed, for example, using biotinylated heterodimers) fluorescent streptavidin (commercially available) can be used to provide a detectable label. A fluorescently labelled multimer is suitable for use in FACS analysis, for example to detect antigen presenting cells carrying the peptide for which the high affinity TCR is specific.

According to the invention, NPM peptides of the invention containing citrulline can be used as targets for cancer immunotherapy via T cell receptors (TCRs). TCRs are designed to recognise short peptide antigens that are displayed on the surface of APCs in complex with MHC molecules (Davis et al. 1998). The identification of particular citrulline containing peptides is advantageous for the development of novel immunotherapies. Such therapeutic TCRs may be used, for example, as soluble targeting agents for the purpose of delivering cytotoxic or immune effector agents to the tumour (Boulter et al. 2003; Liddy et al. 2012; McCormack et al. 2013), or alternatively they may be used to engineer T cells for adoptive therapy (June et al. 2014).

A TCR of the present invention (or multivalent complex thereof) may alternatively or additionally be associated with (e.g. covalently or otherwise linked to) a therapeutic agent which may be, for example, a toxic moiety for use in cell killing, or an immunostimulating agent such as an interleukin or a cytokine. A multivalent high affinity TCR complex of the present invention may have enhanced binding capability for a TCR ligand compared to a non-multimeric wild-type or high affinity T cell receptor heterodimer. Thus, the multivalent high affinity TCR complexes according to the invention are particularly useful for tracking or targeting cells presenting particular antigens in vitro or in vivo, and are also useful as intermediates for the production of further multivalent high affinity TCR complexes having such uses. The high affinity TCR or multivalent high affinity TCR complex may therefore be provided in a pharmaceutically acceptable formulation for use in vivo.

High affinity TCRs may be used in the production of soluble bi-specific reagents. A preferred embodiment is a reagent which comprises a soluble TCR, fused via a linker to an anti-CD3 specific antibody fragment. Further details including how to produce such reagents are described in WO10/133828. TCRs of the invention may be used as therapeutic reagents. In this case the TCRs may be in soluble form and may preferably be fused to an immune effector. Suitable immune effectors include but are not limited to, cytokines, such as IL-2 and IFN-α; superantigens and mutants thereof; chemokines such as IL-8, platelet factor 4, melanoma growth stimulatory protein; antibodies, including fragments, derivatives and variants thereof, that bind to antigens on immune cells such as T cells or NK cell (e.g. anti-CD3, anti-CD28 or anti-CD16); and complement activators.

The binding moiety of the invention may be an antibody. The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term “antibody” includes antibody fragments, derivatives, functional equivalents and homologues of antibodies, humanised antibodies, including any polypeptide comprising an immunoglobulin binding domain, whether natural or wholly or partially synthetic and any polypeptide or protein having a binding domain which is, or is homologous to, an antibody binding domain. Chimeric molecules comprising an immunoglobulin binding domain, or equivalent, fused to another polypeptide are therefore included. Cloning and expression of chimeric antibodies are described in EP-A-0120694 and EP-A-0125023. A humanised antibody may be a modified antibody having the variable regions of a non-human, e.g. murine, antibody and the constant region of a human antibody. Methods for making humanised antibodies are described in, for example, US Patent No. 5225539. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to herein as “mab”.

It is possible to take an antibody, for example a monoclonal antibody, and use recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin (see, for instance, EP-A-184187, GB 2188638A or EP-A-239400). A hybridoma (or other cell that produces antibodies) may be subject to genetic mutation or other changes, which may or may not alter the binding specificity of antibodies produced.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al. 1989) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. 1988; Huston et al. 1988); (viii) bispecific single chain Fv dimers (PCT/US92/09965) and (ix) “diabodies”, multivalent or multispecific fragments constructed by gene fusion (WO94/13804; (Holliger and Winter 1993)). Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804). Where bispecific antibodies are to be used, these may be conventional bispecific antibodies, which can be manufactured in a variety of ways (Holliger and Winter 1993), e.g. prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in (Traunecker, Lanzavecchia, and Karjalainen 1991). Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied, and an antibody of appropriate specificity selected. An “antigen binding domain” is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

Also encompassed within the present invention are binding moieties based on engineered protein scaffolds. Protein scaffolds are derived from stable, soluble, natural protein structures which have been modified to provide a binding site for a target molecule of interest. Examples of engineered protein scaffolds include, but are not limited to, affibodies, which are based on the Z-domain of staphylococcal protein A that provides a binding interface on two of its a-helices (Nygren 2008); anticalins, derived from lipocalins, that incorporate binding sites for small ligands at the open end of a beta-barrel fold (Skerra 2008), nanobodies, and DARPins. Engineered protein scaffolds are typically targeted to bind the same antigenic proteins as antibodies, and are potential therapeutic agents. They may act as inhibitors or antagonists, or as delivery vehicles to target molecules, such as toxins, to a specific tissue in vivo (Gebauer and Skerra 2009). Short peptides may also be used to bind a target protein. Phylomers are natural structured peptides derived from bacterial genomes. Such peptides represent a diverse array of protein structural folds and can be used to inhibit/disrupt protein-protein interactions in vivo (Watt 2006).

As discussed, the inventors have found that certain modified NPM antigens are associated with tumours and citrullinated peptides stimulate T cell responses which can be used to raise an immune response against tumours. The present invention provides an antigen of the first aspect, a complex of the second aspect, and/or a binding moiety of the third aspect for use in medicine. The antigen of the first aspect, complex of the second aspect, and/or binding moiety of the third aspect can be used in a method for treating cancer. Also provided are the use of an antigen of the first aspect, a complex of the second aspect, and/or a binding moiety of the third aspect in the manufacture of a medicament for the treatment of cancer, as well as a method of treating cancer, comprising administering an antigen of the first aspect, a complex of the second aspect, and/or a binding moiety of the third aspect of the invention to a subject in need of such treatment. Antigens in accordance with the present invention may be used alone or in combination as a pool. In addition, they may be used in combination with other therapeutic agents, such as anti-cancer agents including but not limited to checkpoint blockade drugs such as ipilimumab, pembrolizumab and Nivolumab.

The inventors are the first to show that citrullinated NPM peptides can stimulate potent T cell responses. The invention provides suitable means for local stimulation of an immune response directed against tumour tissue in a subject. T cells specific for these NPM cit peptides could target tumour cells to elicit strong anti-tumour effects in vivo, thus providing the first evidence for the use of NPM cit epitopes as vaccine targets for cancer therapy. Stimulation of an immune response directed against a vaccine target includes the natural immune response of the patient and immunotherapeutic treatments aiming to direct the immune response against the tumour (e.g. checkpoint inhibitors, CAR-Ts against tumour antigens and other tumour immunotherapies). Such support or induction of the immune response may in various clinical settings be beneficial in order to initiate and maintain the immune response and evade the tumour-mediated immunosuppression that often blocks this activation. These responses may be tolerised for the treatment of autoimmune diseases.

In some embodiments, the cellular immune response is specific for the stress induced post-translationally modified peptide wherein immune response includes activation of T cells expressing TCRαβ or ʏδ. The present invention also relates to TCRs, individual TCR subunits (alone or in combination), and subdomains thereof, soluble TCRs (sTCRs), for example, soluble αβ dimeric TCRs having at least one disulphide inter-chain bond between constant domain residues that are not present in native TCRs, and cloned TCRs, said TCRs engineered into autologous or allogeneic T cells or T cell progenitor cells, and methods for making same, as well as other cells bearing said TCR.

The cancer may be breast cancer including oestrogen receptor negative breast cancer, colorectal cancer, lung cancer, ovarian cancer renal cancer, liver cancer and AML.

The present invention provides pharmaceutical composition comprising an antigen, complex and/or binding moiety of the present invention be formulated with an adjuvant or other pharmaceutically acceptable vaccine component. In particular embodiments, the adjuvant is a TLR ligand such as CpG (TLR9) MPLA (TLR4), imiquimod (TLR7), poly I:C (TLR3) or amplivant TLR½ ligand, GMCSF, an oil emulsion, a bacterial product or whole inactivated bacteria.

The antigen may be a T or B cell antigen. Peptides in accordance with the present invention may be used alone or in combination. In addition, they may be used in combination with other therapeutic agents, such as anti-cancer agents including but not limited to checkpoint blockade drugs such as ipilimumab.

Antigens in accordance with the invention may be delivered in vivo as a peptide, optionally in the form of a peptide as disclosed in WO02/058728. The inventors have surprisingly found that antigens of the invention give rise to strong immune responses when administered as a peptide. Such peptides may be administered as just the sequence of the peptide, or as a polypeptide containing the antigen, or even as the full-length protein. Alternatively, antigens in accordance with the invention may be administered in vivo as a nucleic acid encoding the antigen, encoding a polypeptide containing the antigen or even encoding the full-length protein. Such nucleic acids may be in the form of a mini gene, i.e. encoding a leader sequence and the antigen or a leader sequence and full-length protein.

As used herein, the term “treatment” includes any regime that can benefit a human or non-human animal. The antigen and/or nucleic acid and/or complex and/or binding moiety may be employed in combination with a pharmaceutically acceptable carrier or carriers to form a pharmaceutical composition. Such carriers may include, but are not limited to, saline, buffered saline, dextrose, liposomes, water, glycerol, ethanol and combinations thereof.

It is envisaged that injections will be the primary route for therapeutic administration of the compositions of the invention although delivery through a catheter or other surgical tubing may also be used. Some suitable routes of administration include intravenous, subcutaneous, intradermal, intraperitoneal and intramuscular administration. Liquid formulations may be utilised after reconstitution from powder formulations.

For intravenous injection, or injection at the site of affliction, the active ingredient will be in the form of a parentally acceptable aqueous solution which is pyrogen-free, has suitable pH, is isotonic and maintains stability. Those of relevant skill in the art are well able to prepare suitable solutions using, for example, isotonic vehicles such as sodium chloride injection, Ringer’s Injection or Lactated Ringer’s Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Where the formulation is a liquid it may be, for example, a physiologic salt solution containing non-phosphate buffer at pH 6.8-7.6, or a lyophilised powder.

The composition may be administered in a localised manner to a tumour site or other desired site or may be delivered in a manner in which it targets tumour or other cells. In some embodiments, the antigen are administered without an adjuvant for a cellular immune response including activation of T cells expressing TCRαβ or ʏδ.

The compositions are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. The compositions of the invention are particularly relevant to the treatment of cancer, and in the prevention of the recurrence of such conditions after initial treatment or surgery. Examples of the techniques and protocols mentioned above can be found in Remington’s Pharmaceutical Sciences (Remington 1980). A composition may be administered alone or in combination with other treatments, either simultaneously or sequentially dependent upon the condition to be treated. Other cancer treatments include other monoclonal antibodies, other chemotherapeutic agents, other radiotherapy techniques or other immunotherapy known in the art. One particular application of the compositions of the invention is as an adjunct to surgery, i.e. to help to reduce the risk of cancer reoccurring after a tumour is removed. The compositions of the present invention may be generated wholly or partly by chemical synthesis. The composition can be readily prepared according to well-established, standard liquid or, preferably, solid-phase peptide synthesis methods, general descriptions of which are broadly available (see, for example, in Solid Phase Peptide Synthesis, 2nd edition (Stewart 1984), in The Practice of Peptide Synthesis (Bodanzsky 1984) and Applied Biosystems 430A User’s Manual, ABI Inc., or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

The antigens, complexes, nucleic acid molecules, vectors, cells and binding moieties of the invention may be non-naturally occurring and/or purified and/or engineered and/or recombinant and/or isolated and/or synthetic.

The invention also provides a method of identifying a binding moiety that binds a complex of the invention, the method comprising contacting a candidate binding moiety with the complex and determining whether the candidate binding moiety binds the complex.

Preferred features of each aspect of the invention are as for each of the other aspects mutatis mutandis. The prior art documents mentioned herein are incorporated to the fullest extent permitted by law.

### EXAMPLES

The present invention will now be described further with reference to the following examples and the accompanying drawings.

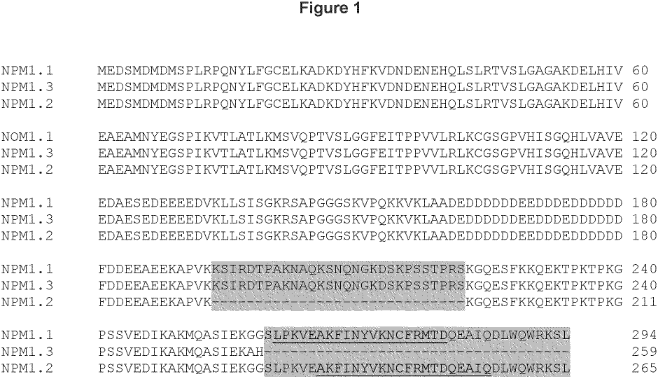

FIG. 1: Sequence alignment of spliced variants of Human Nucleophosmin Alignment of Human nucleophosmin NPM1.1 against two alternatively spliced isoforms (NPM1.2 and NPM1.3). Light grey represents non-homologous regions.

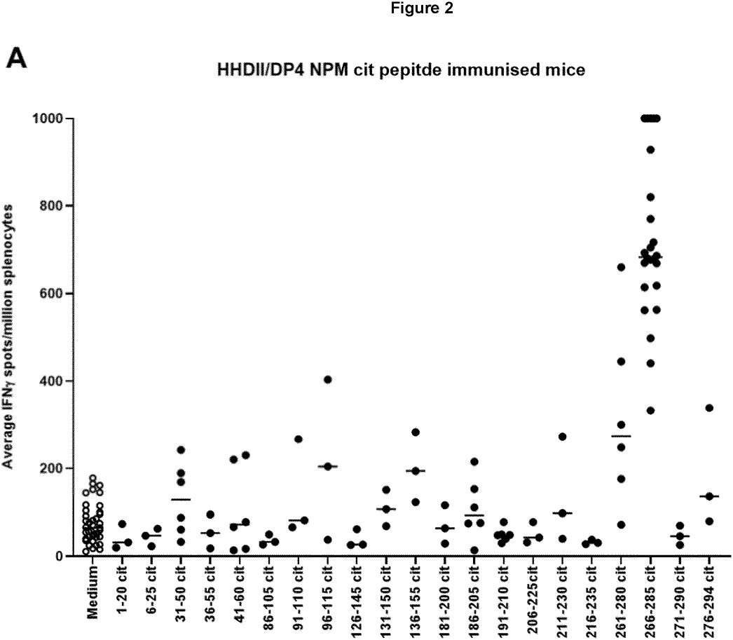

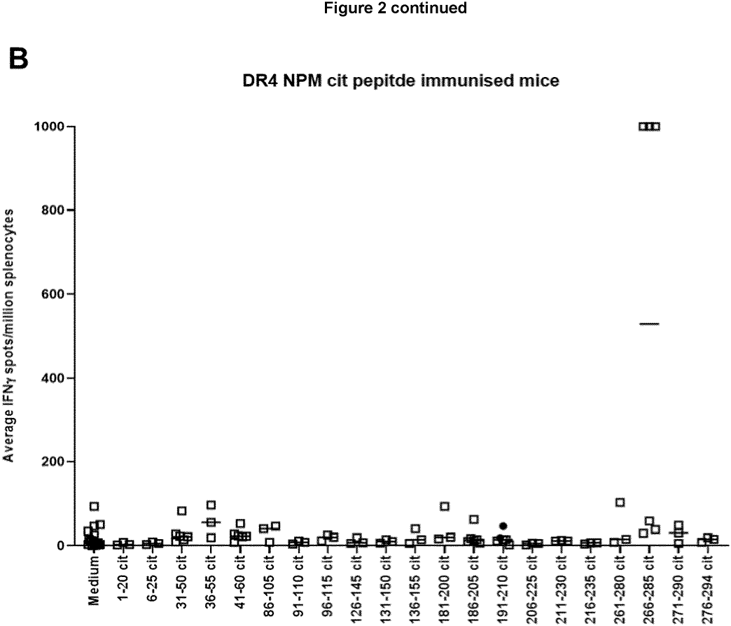

FIG. 2: Screening IFNʏ responses to citrullinated Nucleophosmin peptide pools Transgenic mouse strains expressing HHDII/DP4 or human HLA-DR4 mice were used to screen for IFNy responses to peptide (A and B). Mice were immunised with peptide pools of 4-5 non-overlapping NPM peptides over three weeks. Splenocytes were harvested 21 days after the initial dose was administered. Ex vivo responses to stimulation with human NPM peptides was assessed by IFNy ELISpot. Media only responses were used as a negative control. For each pool n=3 mice. Statistical significance of peptide response was compared to media only responses for each pool and determined using ANOVA with Dunnett’s post-hoc test * p<0.5, ** p<0.01, *** p<0.001, **** p<0.0001.

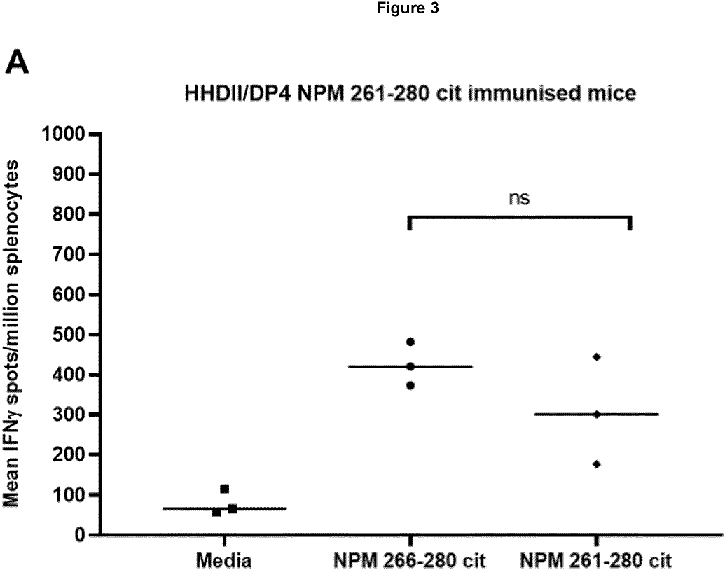

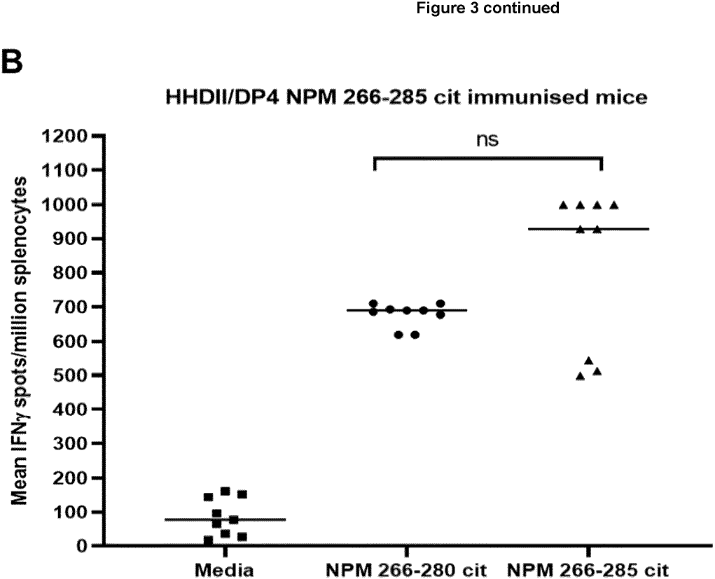

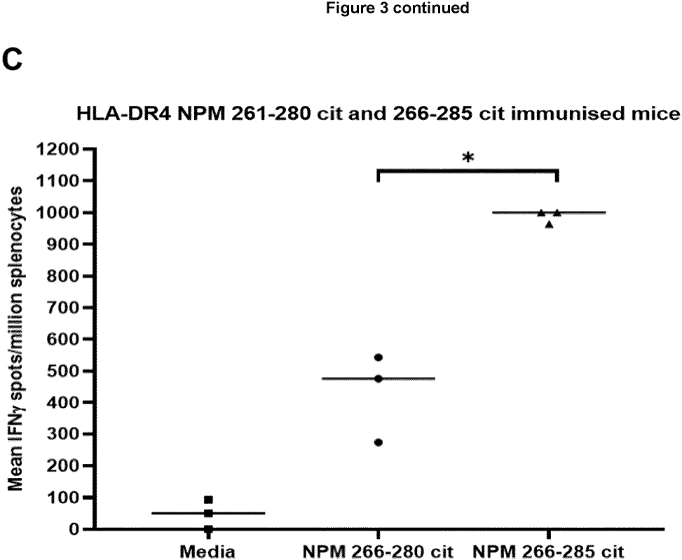

FIG. 3: Defining NPM core epitope that induces strong IFNʏ responses HLA-DP4 (A and B) and HLA-DR4 (C) transgenic mice were immunised with three doses of NPM 261-280 cit (A) or 266-285 cit (B) or a combination of NPM 261-280 cit and NPM 266-285 cit (C) peptide over a course of three weeks, with weekly immunisations. Splenocytes were collected 7 days after the third dose was administered. Ex vivo IFNy ELISpot was performed to determine the response to NPM 261-280 cit, NPM 266-285 cit and NPM 266-280 cit peptides. Statistical significance of peptide response to NPM 266-280 cit was compared to the responses to media alone, NPM 260-280 cit and NPM 266-285 cit using using ANOVA with Dunnett’s post-hoc test * p<0.5, ** p<0.01, *** p<0.001, **** p<0.0001.

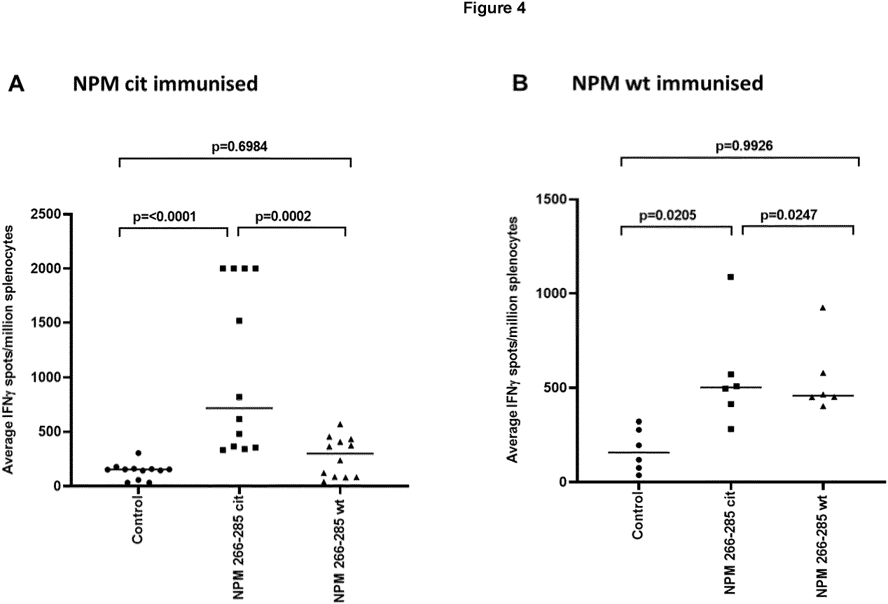

FIG. 4: Human Nucleophosmin 266-285 induces strong IFNʏ and Granzyme B responses Transgenic mice were immunised with three doses of NPM 266-285 cit (A) or 266-285 wt (B) peptide over a course of three weeks, with weekly immunisations. Splenocytes were collected 21 days after the initial dose was administered. Ex vivo IFNy ELISpot was performed to determine the response to NPM 266-285 cit and 266-285 wt. To determine if the responses were mediated by CD4 or CD8 T cells an ex vivo ELISpot was performed in the presence of an anti CD4 or CD8 blocking antibody. Statistical significance of peptide responses was compared using ordinary one-way ANOVA comparison * p<0.5, ** p<0.01, *** p<0.001, **** p<0.0001, NS = not significant. When the ELISpot spot count exceeded 1000 spots a value of 1000 was assigned.

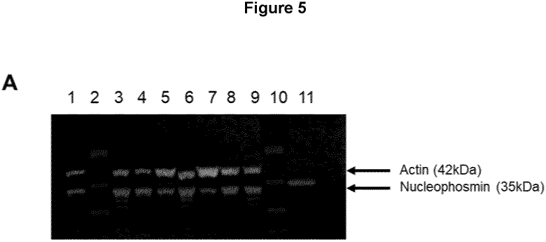

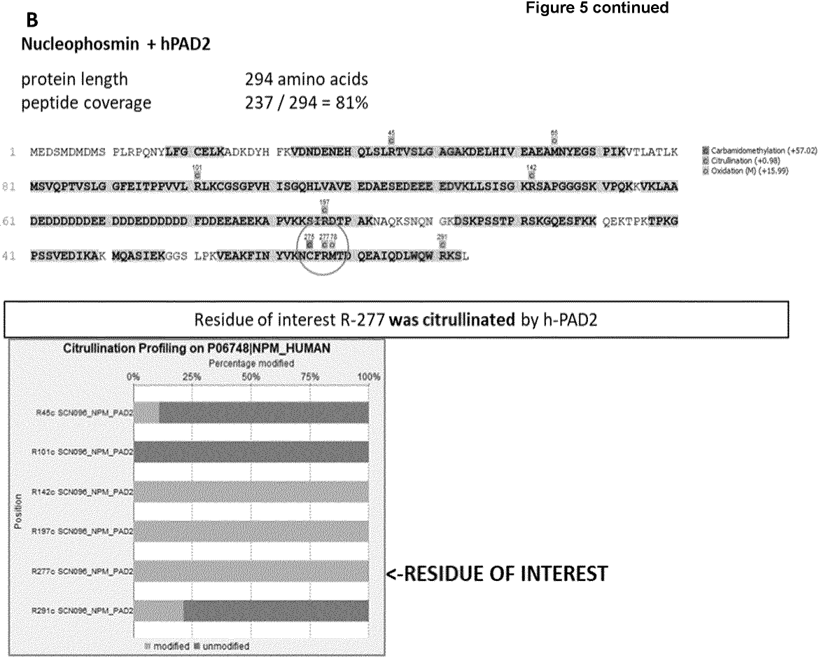

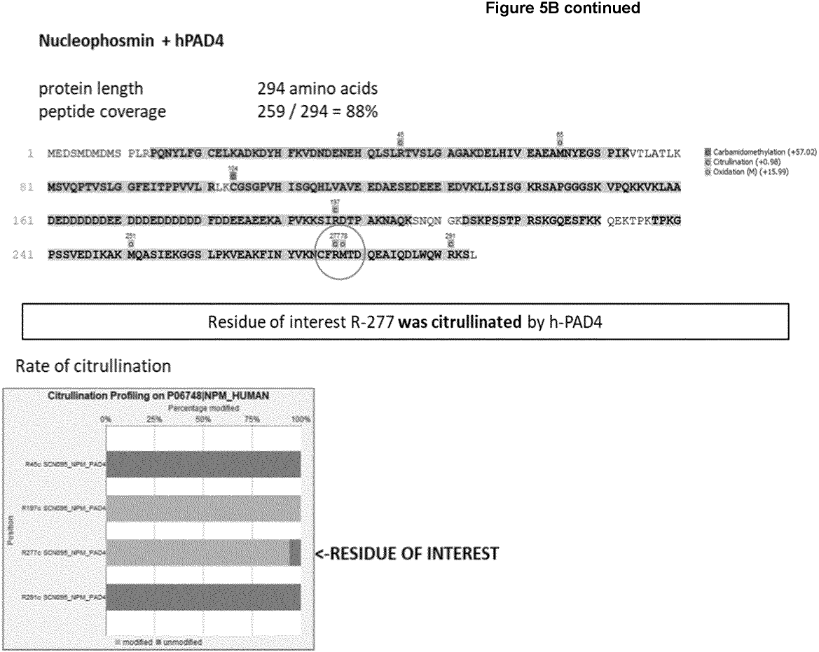

FIG. 5: Expression of NPM on cancer cell lines and detection of NPM 266-285 cit following in vitro citrullination Immunoblot (A) of lysates from HeLa (Lane 1), ladder (Lane 2), PY8119 (Lane 3), PY230 (Lane 4), pan02 (Lane 5), LLC2 (Lane 6), TRAMP (Lane 7), ID8 (Lane 8), B16F1 (Lane 9), ladder (Lane 10), recombinant NPM (Lane 11) cell lines against ladder probed for nucleophosmin (NPM) and β actin. The bands correspond to the expected size for NPM (35 kDa) and β-actin (42 kDa). In vitro citrullination of NPM was performed in the presence of either PAD2 or PAD4, followed by mass spectrometry analysis (B) to identify the sites of citrullination.

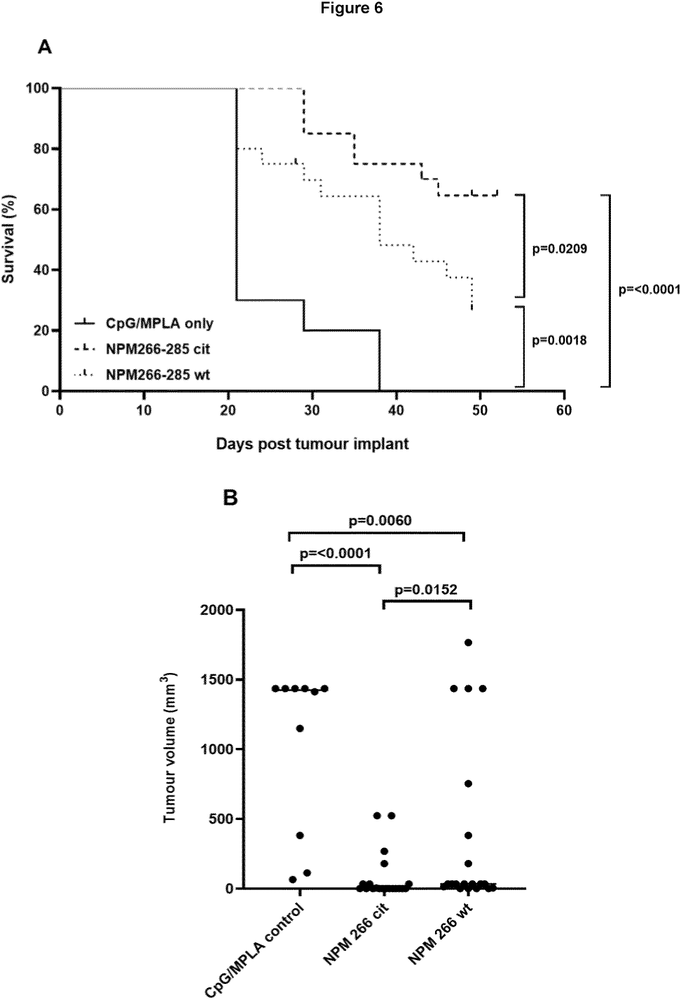

FIG. 6: Human Nucleophosmin 266-285 cit peptide provides an in vivo survival advantage in anti-tumour studies HHDII/DP4 mice were challenged with B16 tumour with IFNy inducible DP4 and 4 days later immunised with NPM266-285 cit or wt peptides. Overall survival (A) and tumour volume (B) at day 24 post tumour implant are shown for control mice (CpG/MPLA only) and mice immunised with either NPM 266-285 cit peptide or NPM 266-285 wt peptide, n=10 in CpG/MPLA control group, n=20 in immunised groups, results are shown from two independent experiments. Statistical differences between immunised and control mice were determined by Mantel-Cox test, p values are shown. For tumour volume medians and p values are shown as determined by Mann Whitney U test.

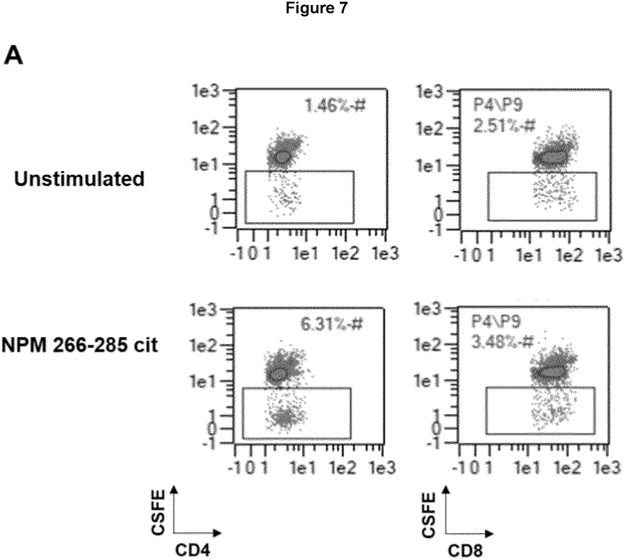

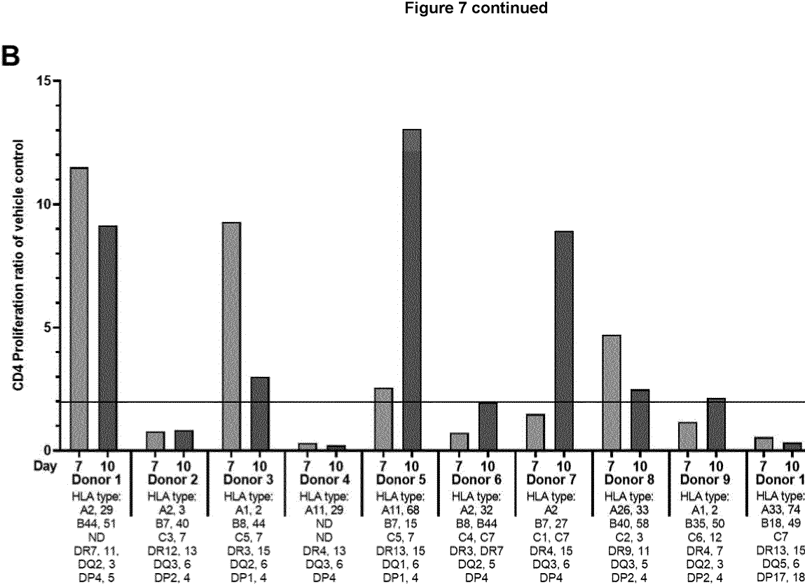

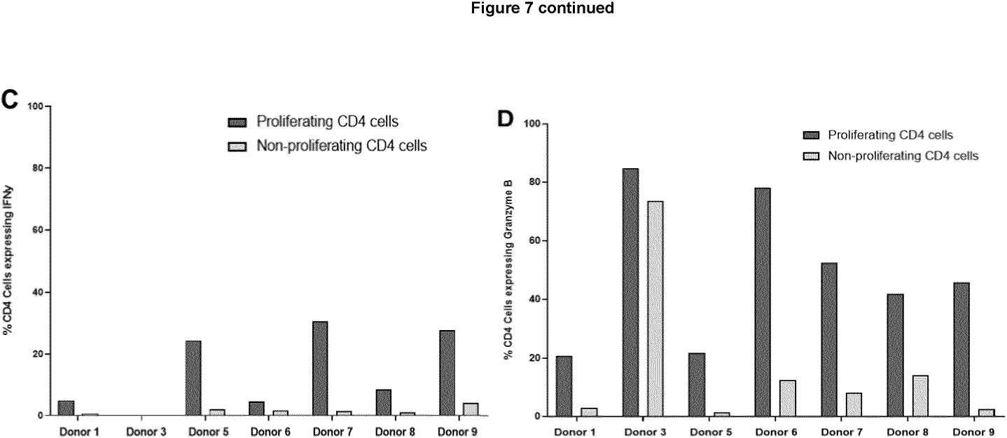

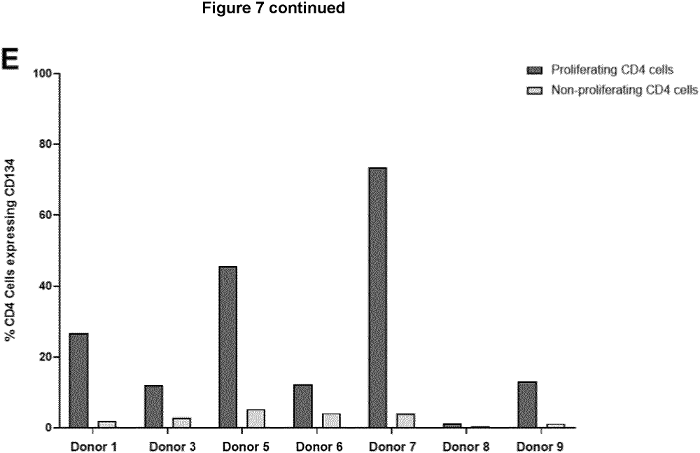

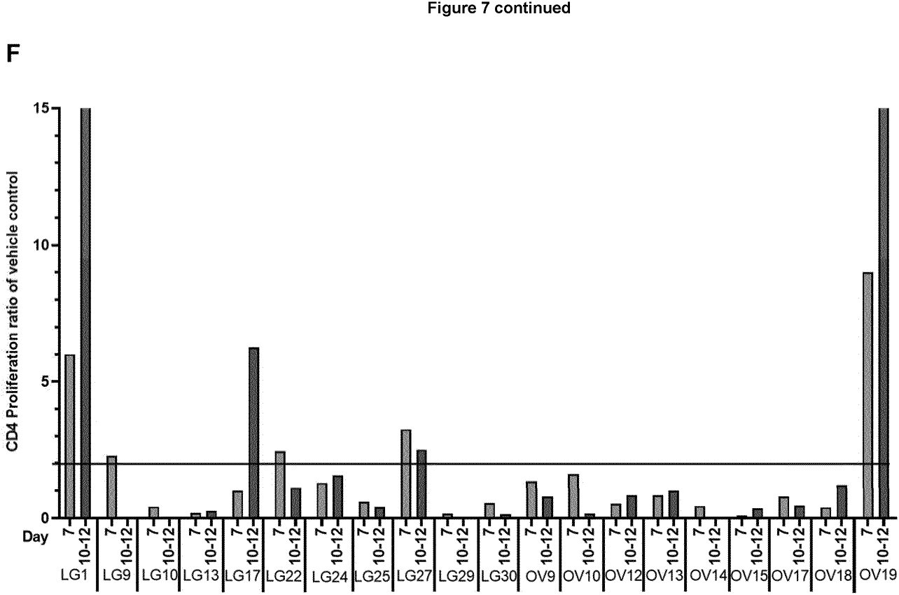

FIG. 7: Human Nucleophosmin 266-285 cit peptide induces responses in human PBMCs PBMCs were isolated from 10 healthy donors, HLA typing was performed on each donor, 9 HLA-DP4 positive donors (2 also express HLA-DR4) and 1 HLA-DP4 negative donors were used. PBMCs isolated from each donor was cultured with media or human NPM 266-285 cit peptide. PMBCs were labelled with CSFE prior to stimulation with NPM 266-285 cit peptide, a representative flow cytometry plot is shown (A). The proliferative responses of CD4 populations within the CSFE labelled cell population was assessed by flow cytometry on days 7 and 10 (B). The ability of proliferating or non-proliferating CD4 cells to express IFNy (C), Granzyme B (D) and CD134 (E) was assessed on days 7 and 10, responses on day 10 are represented.

FIG. 8: Immune response to human Nucleophosmin 266-285 cit peptide is mediated by naive T cells PBMCs isolated from healthy donors were either CD45RO depleted or left non-depleted and cultured with media or human NPM 266-285 cit peptide. PMBCs were labelled with CSFE prior to stimulation with NPM 266-285 cit peptide. The proliferative responses of CD4 populations within the CSFE labelled cell population was assessed by flow cytometry on day 11 (FIG. 8). The T cell responses were compared with non-depleted CD45RO cultures.

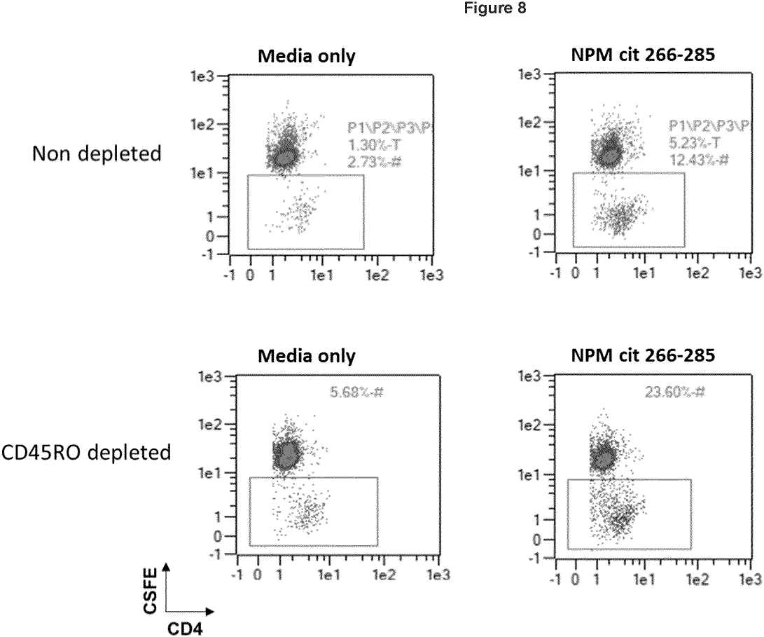

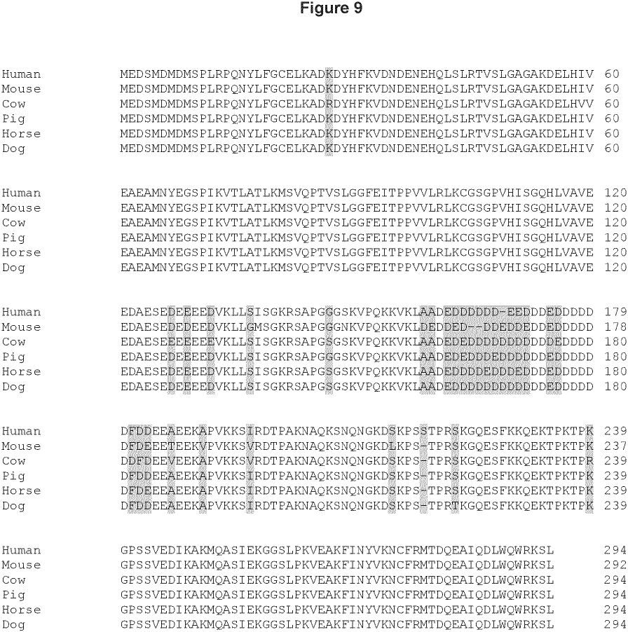

FIG. 9: Nucleophosmin sequence comparison Alignment of human NPM with equivalent sequences from other species (Mouse, Cow, Pig, Sheep, Horse, Dog).

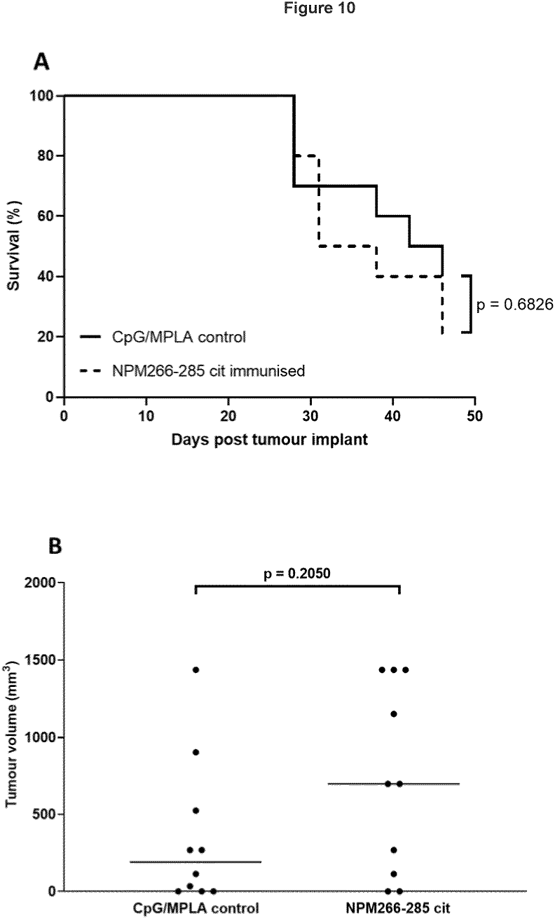

FIG. 10: PAD2 is responsible for citrullinated NPM in vivo To assess if PAD2 is important for citrullination of NPM, HLA-DP4 mice were challenged with B16F1 tumour cells expressing inducible DP4 tumour cells lacking PAD2 enzymes followed by immunisation on day 4, 11 and 18. Tumour growth and survival was monitored and n= 10 mice/group. Overall survival shown on (A) and tumour volumes at day 28 on (B). Statistical differences between immunised and control mice were determined by Mantel-Cox test, p values are shown. For tumour volume medians and p values are shown as determined by Mann Whitney U test.

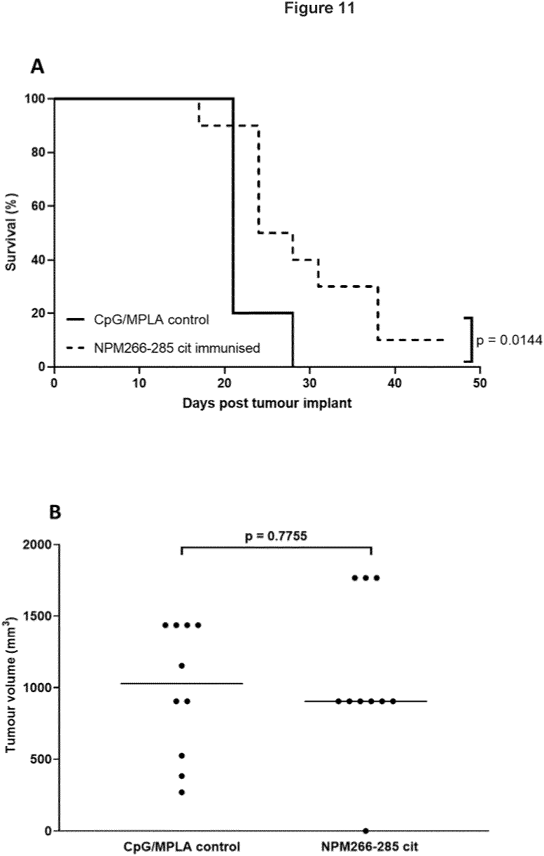

FIG. 11: NPM266-285cit mediated survival advantage is achieved through MHC II To assess if NPM266-285cit is also effective in the absence of MHC II HLA-DP4 mice were challenged with B16F1 tumour cells expressing HHDII but lacking DP4 followed by immunisation on day 4, 11 and 18. Tumour growth and survival was monitored and n= 10 mice/group. Overall survival shown on (A) and tumour volumes at day 28 on (B). Statistical differences between immunised and control mice were determined by Mantel-Cox test, p values are shown. For tumour volume medians and p values are shown as determined by Mann Whitney U test.

### METHODS

### 1.1. Commercial mAbs

Anti-IFNʏ antibody (clone XMG1.2), anti-mouse CD4 (clone GK1.5), anti-mouse CD8 (clone 2.43) and anti-human CD4 (clone OKT-4) were purchased from BioXcell, USA. Anti-human CD134 (clone REA621) and anti-human CD8 (clone REA734) were purchased from Miltenyi, Germany. Anti-human CD4 (clone RPA-T4), anti-human Granzyme B (clone GB11) were purchased from Thermo Fisher Scientific, USA, anti-human IFNʏ (clone E780) was purchased from eBioscience, USA.

### 1.2. Cell Lines

The T-cell/B-cell hybrid cell line T2 stably transfected with functional MHC class II DR4 (DRB1*0401;T2 DR4) has been previously described (Kovats et al. 1997). The murine melanoma B16F1, murine pancreatic pan02 cell lines were obtained from the American Tissue Culture Collection (ATCC) and cultured in RPMI medium 1640 (GIBCO/BRL) supplemented with 10% fetal calf serum (FCS), L-glutamine (2 mM) and sodium bicarbonate buffered unless otherwise stated. The murine transgenic TRAMP cell was obtained from ATCC and cultured in dulbecco’s modified Eagle’s medium with 4 mM L-glutamine adjusted to contain 1.5 g/L sodium bicarbonate and 4.5 g/L glucose supplemented with 0.005 mg/ml bovine insulin and 10 nM dehydroisoandrosterone, 90%; fetal bovine serum, 5%; Nu-Serum IV, 5%. The murine mammary adenocarcinoma cell line PY8119 and PY230 were obtained from ATCC and cultured in Ham’s F12 Kaighn’s medium, 5% FBS, the PY230 cell line was also cultured in the presence of 0.1% MITO+ Serum Extender (Corning). The human cell line HeLa and mouse cell line LLC2 were obtained from ATCC and cultured in Eagle’s Minimum Essential Medium supplemented with 10% fetal calf serum. The ID8 cell line was provided by Dr K. Roby at KUMC University of Kansas, USA and cultured in DMEM supplemented with 10% FCS.

### 1.3. Immunogens

### 1.3.1. Peptides

Peptides >90% purity were synthesized by Peptide Synthetics (Fareham, UK) and stored lyophilised in 0.2 mg aliquots at -80° C. On day of use they were reconstituted to the appropriate concentration in 10% dimethyl formamide.

### 1.4. Plasmids and Transfections

Construction of pVitro 2 chimeric and inducible HLA-DR4 plasmids have been described previously (Brentville et al. 2016; Metheringham et al. 2009). To generate the HHDII plasmid, cDNA was synthesized from total RNA isolated from EL4-HHD cells. This was used as a template to amplify HHD using the forward and reverse primers and sub cloned into pCR2.1. The HHD chain, comprising of a human HLA-A2 leader sequence, the human β2-microglobulin (β2M) molecule covalently linked via a glycine serine linker to the α 1 and 2 domains of human HLA-A*0201 MHC class I molecule and the α3, transmembrane and cytoplasmic domains of the murine H-2Db class I molecule, was then inserted into the EcoRV/Hindlll sites of the mammalian expression vector pCDNA3.1 obtained from Invitrogen.

Endotoxin free plasmid DNA was generated using the endofree Qiagen maxiprep kit (Qiagen, Crawley).

Cell lines were transfected using the Lipofectamine Transfection Reagent (Invitrogen) utilising the protocol previously described (Brentville et al. 2016). B16F1 cells were knocked out for murine MHC-I and/or MHC-II using ZFN technology (Sigma) and transfected with constitutive HLA-DP4 using the pVitro 2 chimeric plasmid. Cells were also transfected with the HHDII plasmid comprising of a human HLA-A2 leader sequence, the human β2-microglobulin (β2M) molecule covalently linked via a glycine serine linker to the α 1 and 2 domains of human HLA-0201 MHC class 1 molecule and the α3, transmembrane and cytoplasmic domains of the murine H-2Db class 1 molecule, where relevant as previously described (Xue et al. 2016). B16F1 HHDII cells were also transfected with the pVITRO2 Human HLA-DP4 plasmid and the IFNy inducible plasmid pDCGAS Human HLA-DP4 is described previously (Brentville et al. 2019).

### 1.6 Western Blots

Cell lysates were prepared in RIPA buffer containing protease inhibitor cocktail (Sigma) and proteins separated on a 4-12% NuPAGE Bis-Tris gel (Invitrogen) followed by transfer onto PVDF membrane. The membrane was blocked for 1 hour with 3%BSA then probed with antibodies to human NPM (clone FC82291, Abcam) 1 in 1000 and β actin (clone AC-15, Sigma) 1 in 15000. Proteins were visualised using thefluorescent secondary antibody IRDye 800RD and IRDye 680RD secondary anti mouse (for β actin). Membranes were imaged using a Licor Odyssey scanner. NPM protein was used as a positive control (ab114194, Abcam).

### 1.7 In Vitro Citrullination

The citrullination of NPM was performed in 0.1 M Tris-HCI pH 7.5 (Fisher), 10 mM CaCl2 (Sigma) and 5 mM DTT (Sigma). Final concentration of solution for was 376 mM Tris-HCI pH 7.5, 3.76 mM CaCl2, 1.88 mM DTT. Samples were incubated with PAD enzymes for 2 hrs at 37° C. before storing at -80° C. overnight or until use. PAD2 enzyme was used at a final concentration of 148 mU and PAD4 at a final concentration of 152 mU. PAD enzymes were purchased from Modiquest at 37 mU/µl hPAD2 and 38 mU/µl hPAD4.

### 1.8 Mass Spectrometry

Samples were prepared by trypsin digest at a ratio of 1:50 trypsin to protein overnight at 37° C. Samples were then dried under vacuum and resuspended in 0.1 % formic acid/5% acetonitrile in LCMS grade water before MS analysis. For MS Analysis, samples were injected via autosampler (Eksigent Ekspert nanoLC 425 LC system utilising a 1-10 µl/min pump module running at 5 µl/min) with a 2 min wash trap/elute configuration onto a YMC Triart C18 column (300um i.d., 3 µm particle size, 15 cm) in a column oven at 35° C. Samples were gradient eluted over an 87 min runtime into a SCI EX 6600 TripleT of mass spectrometer via a Duospray (TurboV) source with a 50 µm electrode. The 6600 was set up in IDA mode (Independent Data Acquisition/Data Dependent Acquisition) for 30 ions per cycle fragmentation. Total cycle time 1.8 s, TOFMS scan 250 ms accumulation; 50 ms for each product ion scan.

Data was analysed using PEAKS Studio 8.0 (Bioinformatic Solutions Inc. Waterloo, Canada) searching the SwissProt human (Uniprot manually annotated/curated) database, 25ppm parent mass error tolerance, 0.1 Da fragment mass error tolerance searching for modifications for citrullination (R), deamidation (NQR), oxidation (M). Sites were identified as a confident modification site with a minimum ion intensity of 5%.

### 1.7 Immunisations

### 1.7.1. Immunisation Protocol

HLA-DR4 mice (Taconic, USA) and the HHDII/HLA-DP4 transgenic strain of mouse as described in patent WO2013/017545 A1 (EMMA repository, France) were used, aged between 8 and 12 weeks, and cared for by the staff at Nottingham Trent University. All work was carried out under a Home Office project licence. Peptides were dissolved in 10% dimethylformamide to 1 mg/mL and then emulsified (a series of dilutions) with the adjuvant CpG and MPLA 6 µg/mouse of each (Invivogen, UK). Peptides (25 µg/mouse) were injected subcutaneously at the base of the tail.

For tumour challenge experiments, mice were challenged with 1×10⁵ B16 HHDII/iDP4, B16F1HHDIIMHCIIKO or B16 HHDII/PAD2KOcDP4 cells subcutaneously on the right flank 3 days before primary immunisation (unless stated otherwise) and then immunised as described above. Tumour growth was monitored at 3-4 days intervals and mice humanely euthanised once tumour reached ≥10 mm in diameter.

### 1.8 Analysis of Immune Responses

### 1.8.1 Isolation and Analysis of Animal Tissue

Spleens were disaggregated and treated with red cell lysis buffer for 2 mins. Tumours were harvested and mechanically disaggregated.

### 1.8.2 Peripheral Blood Mononuclear Cell (PBMC) Isolation

Peripheral blood samples were drawn into lithium heparin tubes (Becton Dickinson) and processed immediately following venepuncture. PBMCs were isolated by density gradient centrifugation using Ficoll-Hypaque. Proliferation and cultured ELISpot assay of PBMCs were performed immediately after isolation.

### 1.8.3 Ex Vivo ELISpot Assay

ELISpot assays were performed using murine IFNy capture and detection reagents according to the manufacturer’s instructions (Mabtech, Sweden). In brief, anti-IFNʏ antibody was coated onto wells of a 96-well Immobilin-P plate. Synthetic peptides (at a variety of concentrations) and 5×10⁵ per well splenocytes were added to the wells of the plate in triplicate. LPS at 5 µg/mL was used as a positive control. Peptide pulsed target cells were added where relevant at 5×10⁴ per well in triplicate and plates incubated for 40 hours at 37° C. After incubation, captured IFNy was detected by a biotinylated anti-IFNʏ antibody and developed with a streptavidin alkaline phosphatase and chromogenic substrate. Lipopolysaccharide (LPS; 5 µg/mL) was used as a positive control. For blocking studies, anti-CD4 blocking antibody (RPA-T4) and anti-CD8 blocking antibody (2.43) from Bioxcell were used at 20 µg/mL. Spots were analysed and counted using an automated plate reader (Cellular Technologies Ltd).

### 1.9 Proliferation Assay

Peripheral blood sample (approx. 50 mL) was drawn into lithium heparin tubes (Becton Dickinson). Samples were maintained at room temperature and processed immediately following venepuncture. PBMCs were isolated by density gradient centrifugation using Ficoll-Hypaque. Proliferation assay of PBMCs were performed immediately after PBMC isolation. The median number of PBMCs routinely derived from healthy donor samples was 1.04 × 10⁶ PBMC/mL whole blood (range: 0.6 × 10⁶- 1.48 × 10⁶ / mL). The median viability as assessed by trypan blue exclusion was 93% (range 90-95%).

Freshly isolated PBMCs were loaded with carboxyfluorescein succinimidyl ester (CFSE) (ThermoFisher). Briefly, a 50 µM stock solution in warm PBS was prepared from a master solution of 5 mM in DMSO. CFSE was rapidly added to PBMCs (5 × 10⁶ cells/mL loading buffer (PBS with 5% v/v heat inactivated FCS)) to achieve a final concentration of 5 µM. PBMCs were incubated at room temperature in the dark for 5 mins after which non-cellular incorporated CFSE was removed by washing twice with excess (x10 v/v volumes) of loading buffer (300 g x 10 mins). Cells were made up in complete media to 1.5 × 10⁶/mL and plated and stimulated with media containing vehicle (negative control), PHA (positive control, final concentration 10 µg/mL) or peptides (10 µg/mL) as described above.

On day 7-11, 500 µL of cells were removed from culture, washed in PBS and stained with 1:50 dilution of anti-CD4 (PE-Cy5, clone RPA-T4, ThermoFisher), anti-CD8 efluor 450, clone RPA-T8, ThermoFisher) and anti-CD134 (PE-Cy7, Clone REA621, Miltenyi). Cells were washed, fixed and permeabilized using intracellular fixation/permeablization buffers (ThermoFisher) according to the manufacturer’s instructions. Intracellular staining for cytokines was performed using a 1:50 dilution of anti-IFNʏ (clone 4S.B3, ThermoFisher) or anti-Granzyme B (PE, Clone GB11, Thermofisher). Stained samples were analysed on a MACSQuant 10 flow cytometer equipped with MACSQuant software version 2.8.168.16380 using stained vehicle stimulated controls to determine suitable gates.

### 1.10 FACS Cell Sorting

On day 10, the contents of the culture wells were mixed gently, pooled (according to peptide stimulation) and washed in PBS (300 g x 10mins). Pellets were gently re-suspended in 500µL of PBS containing 10µl of anti CD4 eFluo450 (clone RPA-T4, ThermoFisher, cat no 48-0049-42) and 10µL of anti-CD8 APC (clone RPA-T8, ThermoFisher, cat 17-0088-41). Cells were stained at 4oC for 30 mins before being washed (5 min x 300 g) in 1.0ml of PBS and resuspended in 300µl of FACS sorting buffer (PBS supplemented with 1 mM EDTA, 25 mM HEPES and 1 %v/v HI FCS). 10µl of sample was removed from each stained sample and 90µl of FACS sorting buffer added. 10,000 events were collected on a MACSQuant Analyser 10 flow cytometer to determine proliferation. The remaining cells were used for bulk FACS sorting.

Cells are sorted using sterile conditions in a MoFlo XDP High Speed Cell Sorter machine. All samples are sorted into 1.0 ml of RNA protect (5 parts Protect, Qiagen: 1 part FACS sorting buffer, Sigma) separating the CD4+ve/CFSEhigh and CD4+ve/CFSEIow populations. Sorted cells (bulk) are stored at -80° C.

Determination of the α and β chain pairing of TCRs recognising NPM peptides containing citrulline. Sorted cells (bulk) from CD4+ve/CFSEhigh and CD4+ve/CFSEIow populations in RNA protect are shipped to iRepertoire Inc (Huntsville, AL, USA) for NGS sequencing of the TCRA and TCRB chain to confirm expansion of TCR’s in the CD4+ve/CFSElow cells, proliferating to the peptide in contrast to the non-proliferating CD4+ve/CFSEhigh population. In brief RNA is purified from sorted cells, RT-PCR is performed, cDNA is then subjected to Amplicon rescued multiplex PCR (ARM-PCR) using human TCR α and β 250 PER primers (iRepertoire Inc., Huntsville, AL, USA). Information about the primers can be found in the United States Patent and Trademark Office (Patent Nos. 7,999,092 and 9,012,148B2). After assessment of PCR/DNA samples, 10 sample libraries were pooled and sequenced using the Illumina MiSeq platform (Illumina, San Diego, CA, USA). The raw data was analysed using IRweb software (iRepertoire). V, D, and J gene usage and CDR3 sequences were identified and assigned and tree maps generated using iRweb tools. Tree maps show each unique CDR3 as a coloured rectangle, the size of each rectangle corresponds to each CDR3 abundance within the repertoire and the positioning is determined by the V region usage.

To elucidate the cognate pairing and sequencing of TCRα and TCRβ chains IRepertoire use their iPairTM technology, CD4+ve/CFSElow populations of cells (bulk sorted, that were simultaneously bulk sequenced) are seeded at 1 cell/well into an iCapture 96 well plate. RT-PCR is performed and the TCRα and β chains can be amplified from the single cells using Amplicon rescued multiplex PCR (arm-PCR). Data can be analysed utilising the iPair ™ Software program for frequency of specific chain pairing and the sequences ranked on comparison to bulk data.

### 1.11 Knock Out of PAD2

PAD2 knock out of B16F1 cells was performed by Sigma-Aldrich Cell Design Studio™. CompoZr zinc finger nuclease (ZFN) technology was used targeting NPM exon 1 with pair sequences NM008812-r649a1: CTGCAGCCGCACGGTCCGTTCCCGCAGC and NM008812-656a1: TGGGAGCCGCGTGGAGGCGGTGTACGTG. Following several rounds of retargeting, 89% cutting was achieved and single cell cloning was then performed to establish a stable clone. ddPCR and flow cytometry (ab50257 & ab150063, Abcam) were used by Sigma-Aldrich Cell Design Studio ™ to assess the knockout of PAD2 of the clone. The primers and probes used for ddPCR were from Thermo fisher proprietary (Mm00447012_m1 & Mm00447020_m1).

### 1.12 Statistical Methods

Data were expressed as the number of spots per million splenocytes. Means and standard deviations (SD) were calculated from the quadruplicate readings. Means and SDs were also calculated for each group of three mice. Where appropriate Anova analysis was performed using GraphPad Prism 6 software.

### Example 1 - Sequence Alignment and Homology of Nucleophosmin

In mammals the most prevalent form of NPM is NPM1.1, two alternatively spliced isoforms exist (NPM1.2 and NPM1.3), these are shorter versions of NPM but share a high degree of homology (FIG. 1).

### Example 2 - T Cell Responses in HHDII/DP4 and HHDII/DR4 Mice to Nucleophosmin Epitopes

T cell responses to tumour associated epitopes are often weak or non-existent due to tolerance and T cell deletion within the thymus. The citrullinated NPM peptides were screened in HLA-DR4 and HHDII/DP4 transgenic mice for their ability to stimulate IFNy responses. Every peptide containing an arginine was selected and the arginine residue was replaced with citrulline (cit). The selected peptides are summarised in Table 2.

### Screening of Nucleophosmin Peptide Responses

Screening was performed to identify potential citrullinated NPM epitopes that could generate an immune response in mice. Mice were immunised with pools of 4-6 human citrullinated peptides. To reduce the effect of possible cross reactivity, the peptides within each pool were chosen so that they did not contain any overlapping amino acid sequences. Each pool was administered subcutaneously as a single immunisation given once a week for three weeks. Each peptide pool contained 25 µg of each peptide in combination with CpG/MPLA as an adjuvant. Mice were culled 7 days after the third immunisation, the immune response to each peptide within the immunising pool were assessed by ex vivo IFNy ELISpot (FIG. 2). We have previously shown that citrullinated peptides can induce responses in the transgenic DR4 mouse strain. Given that different mouse strains have different MHC repertoires, two different transgenic strains (DR4 and DP4) were used for screening.

Significant IFNy responses were detected to human NPM citrullinated peptides in HHDII/DP4 and HLA-DR4 transgenic mice (FIG. 2). In the HHDII/DP4 mice (A) the NPM citrullinated peptides 261-280 and 266-285 induced significant immune responses (p=<0.0001 and p=<0.0001 respectively). In contrast, the known citrullinated position at aa197 (B cell epitope) encompassed by peptide 186-205 and 191-210 failed to induce an immune response. In the HLA-DR4 mice, significant IFNy responses (B) were only detected in response to the NPM citrullinated peptide 266-285 (p=<0.0001), no other responses were detected against any other NPM peptides in these mice. There was also no detectable immune response generated to the described B cell epitope contained a citrullinated residue at position 197 (Tanikawa et al. 2009).

TABLE 2

Nucleophosmin peptide utilised

| Coordinates | Sequence | DP4 predicted cores | DR4 predicted cores | T cell response |
|---|---|---|---|---|
| 1-20 | MEDSMDMDMSPL-cit-PQNYLFG | PLRPQNYLF MDMSPLRPQ | MDMDMSPLR LRPQNYLFG | No |
| 6-25 | DMDMSPL-cit-PQNYLFGCELKA | LRPQNYLFG | LRPQNYLFG | No |
| 31-50 | FKVDNDENEHQLSL-cit-TVSLG | QLSLRTVSL | QLSLRTVSL | No |
| 36-55 | DENEHQLSL-cit-TVSLGAGAKD | QLSLRTVSL | QLSLRTVSL LSLRTVSLG | No |
| 41-60 | QLSL-cit-TVSLGAGAKDELHIV | QLSLRTVSL | QLSLRTVSL LRTVSLGAG LSLRTVSLG | No |
| 86-105 | TVSLGGFEITPPVVL-cit-LKCG | None | None | No |
| 91-110 | GFETPPVVL-cit-LKCGSGPVH | None | None | No |
| 96-115 | PPVVL-cit-LKCGSGPVHISGQH | RLKCGSGPV | VLRLKCGSG | No |
| 126-145 | EDEEEEDVKLLSISGK-cit-SAP | LLSISGKRS | LLSISGKRS | No |
| 131-150 | EDVKLLSISGK-cit-SAPGGGSK | LLSISGKRS ISGKRSAPG | LLSISGKRS | No |
| 136-155 | LSISGK-cit-SAPGGGSKVPQKK | ISGKRSAPG | ISGKRSAPG LSISGKRSA | No |
| 181-200 | FDDEEAEEKAPVKKSI-cit-DTP | VKKSIRDTP | VKKSIRDTP | No |
| 186-205 | AEEKAPVKKSI-cit-DTPAKNAQ | RDTPAKNAQ IRDTPAKNA | IRDTPAKNA | No |
| 191-210 | PVKKSI-cit-DTPAKNAQKSNQN | IRDTPAKNA | IRDTPAKNA | No |
| 206-225 | KSNQNGKDSKPSSTP-cit-SKGQ | SSTPRSKGQ PSSTPRSKG | SKPSSTPRS | No |
| 211-230 | GKDSKPSSTP-cit-SKGQESFKK | RSKGQESFK PRSKGQESF | SKPSSTPRS | No |
| 216-235 | PSSTP-cit-SKGQESFKKQEKTP | RSKGQESFK | PSSTPRSKG | No |
| 261-280 | LPKVEAKFI NYVKNCF-cit-MTD | YVKNCFRMT | INYVKNCFR | Yes |
| 266-280 | AKFINYVKNCF-cit-MTD* | | | Yes |
| 266-285 | AKFINYVKNCF-cit-MTDQEAIQ | FRMTDQEAI | FRMTDQEAI | Yes |
| 271-290 | YVKNCF-cit-MTDQEAI QDLWQW | CFRMTDQEA FRMTDQEAI | YVKNCFRMT FRMTDQEAI | No |
| 276-294 | F-cit-MTDQEAIQDLWQWcitKSL | FRMTDQEAI | FRMTDQEAI | No |

The two peptides, 261-280 cit and 266-285 cit, share a common 15 amino acid epitope, these peptides generated high frequency IFNy responses in HHDII/DP4 and HLA-DR4 mice (FIGS. 2A & 2B). When aligned,

(261-280) LPKVEAKFINYVKNCF-cit-MTD
(266-285) AKFINYVKNCF-cit-MTDQEAIQ

the core epitope for responses in both HLA-DR4 and HHDII/DP4 mice must lie within the sequence:

(266-280) AKFINYVKNCF-cit-MTD

The core epitope was confirmed by immunising mice once weekly for three weeks with 25 µg of the NPM 261-280 cit or 266-285 cit peptide. HHDII/DP4 mice were immunised with the individual peptides whereas HLA-DR4 mice were immunised with a combination of NPM 261-280 cit and 266-285 cit, all given with CpG/MPLA. Mice were culled 7 days after the third immunisation. The immune response to NPM 261-280 cit and NPM 266-285 cit was assessed by ex vivo IFNy ELISpot alongside the response to NPM 266-280 cit, the suggested core epitope (FIG. 3). Significant IFNʏ responses were detected to human NPM 261-280 cit and NPM 266-285 cit peptides in HHDII/DP4 and to NPM 266-285 cit peptide in HLA-DR4 transgenic mice. There was no significant difference between the response to the core peptide NPM 266-280 cit and NPM 261-280 cit or NPM 266-285 cit in HHDII/DP4 transgenic mice. There was a significant difference in the response to NPM 266-280 cit and NPM 266-285 cit in HLA-DR4 transgenic mice, indicating that in HLA-DR4 mice the core peptide is different and possibly includes amino acids QEAIQ (position 281-285).

The 266-285 cit peptide generated the strongest immune response in both HLA-DR4 and HHDII/DP4 mice (FIGS. 2A and 2B). Further investigations focused on this peptide.

To determine if the immune response to the NPM 266-285 peptide is specific to the citrullinated peptide and not the wild type (wt) version, mice were immunised with NPM 266-285 cit or NPM 266-285 wt peptides. HHDII/DP4 mice received 25 µg peptide (NPM 266-285 cit or NPM 266-285 wt) subcutaneously once a week for three weeks. Mice were culled 7 days after the third immunisation, the immune response to each peptide was assessed by ex vivo ELISpot (FIGS. 4A and 4B). Low to moderate IFNy responses were detected in mice immunised with NPM 266-285 wt and the response cross reacted with the citrullinated peptide (FIG. 4B), these responses were not specific to the NPM 266-285 wt peptide as similar responses were seen when cells were stimulated with the NPM 266-285 cit peptide. In contrast, strong IFNy responses were detected in mice immunised with NPM 266-285 cit, these responses were significant when compared to the response to the NPM 266-285 wt peptide (p=0.0002) and the control (p=<0.0001). This confirmed that the immune response generated in HHDII/DP4 mice is in response to the citrullinated version of the NPM 266-285 peptide.

### Example 3 - Cit Nucleophosmin Peptide Presented on Tumour Cells Can Be Targeted For Tumour Therapy

The inventors had already established by Western blotting that the melanoma B16F1 cell lines constitutively express NPM and in vitro citrullination of NPM generates citrulline at position 277 (FIG. 5A and B). Next, the anti-tumour effect of NPM 266-285 cit peptide immunisation was assessed in vivo. The effect of immunisation with NPM 266-285 (cit and wt) on the growth of the mouse B16 melanoma cell line transfected with IFNy inducible human DP4 (iDP4) was assessed. Mice were challenged with B16 HHDII/iDP4 tumour cells 3 days prior to immunisation with NPM 266-285 wt or NPM 266-285 cit. Mice immunised with NPM 266-285 cit peptide showed a significant survival advantage over control mice immunised with CpG/MPLA only (FIG. 6A). Control mice showed 0% survival at 50 days whereas NPM 266-285 cit immunised mice showed 65% survival (p=<0.0001), mice immunised with 266-285 wt also showed a significant 30% survival (p=0.0018) suggesting again that the T cells stimulated by the wild type peptide can cross react with the citrullinated epitope and recognise tumours. There was no associated toxicity. The tumour volumes at day 24 post tumour implant was also significantly lower (p=<0.0001) in the NPM 266-285 cit immunised mice (FIG. 6B, median 0 mm³) compared to the control group (median 1425 mm³). The tumour volume was also significantly lower (p=0.0152) in the NPM 266-285 wt immunised mice (median 34 mm³) compared to the control group (median 1425 mm³).

### Example 4 - Responses to NPM in Healthy Human Donors and Cancer Patients

In HHDII/iDP4 mice, the response to NPM 266-285 cit peptide could not be detected 2 days post immunisation, but could be detected 12 days after immunisation. This suggests that these are naive responses and no pre-existing immunity exists in these mice. This raised the question of whether humans have or can generate immune responses to NPM 266-285 cit peptide. To investigate this, PBMC’s were isolated from ten healthy donors and cultured in the presence of NPM 266-285 cit peptide. Nine donors were HLA-DP4 positive, two of these were also HLA-DR4 positive, an additional donor (donor 10) was HLA-DP4 and HLA-DR4 negative (negative control).

PBMCs from ten healthy donors were labelled with Carboxyfluorescein succinimidyl ester (CFSE) prior to in vitro culture in the presence of NPM 266-285 cit peptide. On day 7 and 10 cells were stained with anti-CD4 and anti-CD8 fluorochome conjugated antibodies, proliferation was then assessed by flow cytometry (FIG. 7A). On day 7, a CD4 NPM 266-285 specific proliferating (CFSE^{low}) population could be detected in four out of ten donors (nine are HLA-DP4 positive donors), this increased at day 10 with seven out of ten donors (nine are HLA-DP4 positive donors) showing a specific response (FIG. 7B). On day 10, functional analysis was performed on the seven donors that showed a good CD4 NPM 266-285 specific proliferative response. The expression of IFNy, Granzyme B and CD134 was determined for all donors, comparing the proliferating and non-proliferating CD4 T cells (FIG. 7C, D and E). The proliferating CD4 NPM 266-285 specific T cells from all seven donors expressed granzyme B. In addition; six out of seven donors also expressed IFNy and CD134, this expression was only associated with the proliferating T cells in the majority of donors (six out of seven).

PBMCs from eleven cancer patients and nine ovarian cancer patients were labelled with Carboxyfluorescein succinimidyl ester (CFSE) prior to in vitro culture in the presence of NPM 266-285 cit peptide. On day 7 and 10, cells were stained with anti-CD4 and anti-CD8 fluorochome conjugated antibodies, proliferation was then assessed by flow cytometry (FIG. 7F). On day 7, a CD4 NPM 266-285 cit specific proliferating (CFSE^{low}) population could be detected in four out of eleven lung cancer patients. This decreased at day 10 with only three out of eleven patients showing a specific response (FIG. 7F). On day 7, CD4 NPM 266-285 cit specific proliferating (CFSE^{low}) population could be detected in one ovarian cancer patient out of nine. This remained the same on day 10 with only the one patient showing a specific response to 266-285 NPM cit peptide.

These results suggest that the majority of heathy donors are able to generate a CD4 proliferative response to NPM 266-285 cit peptide which is also associated with the upregulation of functional markers associated with cytotoxic activity. PBMCs from some cancer patients are able to develop a CD4 response to NPM 266-285 cit peptide, but this is lower than the number of responding healthy donors. This lower frequency maybe due to medication or some degree of tumour mediated immune suppression in these patients.

### Example 5 - In Healthy Human Donors Naive T Cell Populations Respond to NPM Cit Peptide

PBMCs were isolated from two healthy donors, and split into two fractions, CD45RO cells were depleted from one fraction and the second fraction were left non-depleted. PBMCs were labelled with Carboxyfluorescein succinimidyl ester (CFSE) prior to in vitro culture in the presence of NPM 266-285 cit peptide. On day 11, cells were stained with anti-CD4 and anti-CD8 fluorochrome conjugated antibodies, proliferation was then assessed by flow cytometry (FIG. 8). On day 11, a CD4 NPM 266-285 cit specific proliferating (CFSE^{low}) population could be detected in both the CD45RO depleted and non-depleted populations. The percentage of proliferating CD4 T cells was higher (23.6%) in the CD45RO depleted population indicating that the naive T cells are responding to the NPM cit peptide.

### Example 6 - Homology of Nucleophosmin Between Different Species

Nucleophosmin is highly conserved between, mouse, dog, sheep, cows, horse, pig and humans (FIG. 9). As the vaccine induces T cell responses in humans and mice, and anti-tumour responses in mice, it can be assumed similar responses will be seen in other species.

### Example 7 - PAD2 Is Responsible for Citrullination of Arginine 277 in Tumours in Vivo

To determine whether the PAD2 or PAD4 enzyme is responsible for the citrullination in vivo, a B16 tumour cell line that lacks PAD2 (B16F1cDP4PAD2KO) was generated. Knocking out the PAD4 enzyme was unsuccessful with cells failing to grow following the knockout of PAD4. Transgenic HLA-DP4 mice were implanted with B16F1cDP4PADKO tumour cells that lacked the PAD2 enzyme. Tumour growth was assessed following immunisation with NPM266-285cit given in combination with CPG/MPLA and compared to tumour growth in a CPG/MPLA control group (FIG. 10). Overall there was no significant survival advantage in B16F1cDP4PADKO tumour bearing mice immunised with NPM266cit when compared to the unimmunised CPG/MPLA control group (p = 0.6826, FIG. 10A). The tumour volumes were calculated on day 28 (FIG. 10B) and there was no significant difference (p=0.2050) between CpG/MPLA control mice (median 190.5 mm³) and NPM266-285 cit (median 696.6 mm³) immunised mice. The anti-tumour responses were lost in B16F1cDP4PADKO tumour bearing mice, demonstrating that PAD2 is critical for the citrullination of arginine 277 in the tumour cells in vivo and for the anti-tumour effects.

### Example 8 - MHC Class II Is Essential For Anti-Tumour Responses Following Immunisation With NPM 266-285 Cit

To determine if MHC class II is essential for the anti-tumour responses observed in tumour bearing mice following immunisation with NPM 266-285 cit, the B16 cell line was engineered where MHC-II had been knocked out (B16F1HHDIIMHCIIKO). Transgenic HHDII mice were implanted with B16F1HHDIIMHCIIKO tumour cells that lack MHC-II. Tumour growth was assessed following immunisation with NPM266-285cit given in combination with CPG/MPLA and compared to tumour growth in a CPG/MPLA control group (FIG. 11). Overall, there was a 10% improvement in overall survival in B16F1HHDIIMHCIIKO tumour bearing mice immunised with NPM 266-285 cit when compared to the CPG/MPLA control group (p = 0.0144, FIG. 11A). The tumour volumes were calculated on 28 day (FIG. 11B) and there was no significant difference (p=0.2050) between CpG/MPLA control mice (median 1027 mm³) and NPM266-285 cit (median 904.3 mm³) immunised mice. The small improvement in survival in MHC-II KO tumours suggests that the CD4 T cells can induce anti-tumour responses by bystander effect by improving CD8 and/or NK responses but the superior anti-tumour responses when tumour express MHC-II suggests that the CD4 T cells can also mediate direct tumour killing.

### REFERENCES

Altschul, S. F., W. Gish, W. Miller, E. W. Myers, and D. J. Lipman. 1990. ‘Basic local alignment search tool’, J Mol Biol, 215: 403-10.

Altschul, S. F., T. L. Madden, A. A. Schaffer, J. Zhang, Z. Zhang, W. Miller, and D. J. Lipman. 1997. ‘Gapped BLAST and PSI-BLAST: a new generation of protein database search programs’, Nucleic Acids Res, 25: 3389-402.

Andreatta, M., V. I. Jurtz, T. Kaever, A. Sette, B. Peters, and M. Nielsen. 2017. ‘Machine learning reveals a non-canonical mode of peptide binding to MHC class II molecules’, Immunology, 152: 255-64.

Arnold, P. Y., N. L. La Gruta, T. Miller, K. M. Vignali, P. S. Adams, D. L. Woodland, and D. A. Vignali. 2002. ‘The majority of immunogenic epitopes generate CD4+ T cells that are dependent on MHC class II-bound peptide-flanking residues’, J Immunol, 169: 739-49.

Arstila, T. P., A. Casrouge, V. Baron, J. Even, J. Kanellopoulos, and P. Kourilsky. 1999. ‘A direct estimate of the human alphabeta T cell receptor diversity’, Science, 286: 958-61.

Ausubel, JM. 1992. Short protocols in molecular biology (John Wiley & Sons).

Bernard, K., E. Litman, J. L. Fitzpatrick, Y. G. Shellman, G. Argast, K. Polvinen, A. D. Everett, K. Fukasawa, D. A. Norris, N. G. Ahn, and K. A. Resing. 2003. ‘Functional proteomic analysis of melanoma progression’, Cancer Res, 63: 6716-25.

Bird, R. E., K. D. Hardman, J. W. Jacobson, S. Johnson, B. M. Kaufman, S. M. Lee, T. Lee, S. H. Pope, G. S. Riordan, and M. Whitlow. 1988. ‘Single-chain antigen-binding proteins’, Science, 242: 423-6.

Bobisse, S., R. Genolet, A. Roberti, J. L. Tanyi, J. Racle, B. J. Stevenson, C. Iseli, A. Michel, M. A. Le Bitoux, P. Guillaume, J. Schmidt, V. Bianchi, D. Dangaj, C. Fenwick, L. Derre, I. Xenarios, O. Michielin, P. Romero, D. S. Monos, V. Zoete, D. Gfeller, L. E. Kandalaft, G. Coukos, and A. Harari. 2018. ‘Sensitive and frequent identification of high avidity neo-epitope specific CD8 (+) T cells in immunotherapy-naive ovarian cancer’, Nat Commun, 9: 1092.

Bodanzsky, Bodanzsky &. 1984. The practice of peptide synthesis (Springer Verlag: New York).

Borer, R. A., C. F. Lehner, H. M. Eppenberger, and E. A. Nigg. 1989. ‘Major nucleolar proteins shuttle between nucleus and cytoplasm’, Cell, 56: 379-90.

Boulter, J. M., M. Glick, P. T. Todorov, E. Baston, M. Sami, P. Rizkallah, and B. K. Jakobsen. 2003. ‘Stable, soluble T-cell receptor molecules for crystallization and therapeutics’, Protein Eng, 16: 707-11.

Brentville, V. A., R. L. Metheringham, B. Gunn, P. Symonds, I. Daniels, M. Gijon, K. Cook, W. Xue, and L. G. Durrant. 2016. ‘Citrullinated Vimentin Presented on MHC-II in Tumor Cells Is a Target for CD4+ T-Cell-Mediated Antitumor Immunity’, Cancer Res, 76: 548-60.

Brentville, V. A., P. Symonds, K. W. Cook, I. Daniels, T. Pitt, M. Gijon, P. Vaghela, W. Xue, S. Shah, R. L. Metheringham, and L. G. Durrant. 2019. ‘T cell repertoire to citrullinated self-peptides in healthy humans is not confined to the HLA-DR SE alleles; Targeting of citrullinated self-peptides presented by HLA-DP4 for tumour therapy’, Oncoimmunology, 8: e1576490.

Brown, J. H., T. S. Jardetzky, J. C. Gorga, L. J. Stern, R. G. Urban, J. L. Strominger, and D. C. Wiley. 1993. ‘Three-dimensional structure of the human class II histocompatibility antigen HLA-DR1’, Nature, 364: 33-9.

Carson, R. T., K. M. Vignali, D. L. Woodland, and D. A. Vignali. 1997. ‘T cell receptor recognition of MHC class II-bound peptide flanking residues enhances immunogenicity and results in altered TCR V region usage’, Immunity, 7: 387-99.

Chan, W. Y., Q. R. Liu, J. Borjigin, H. Busch, O. M. Rennert, L. A. Tease, and P. K. Chan. 1989. ‘Characterization of the cDNA encoding human nucleophosmin and studies of its role in normal and abnormal growth’, Biochemistry, 28: 1033-9.

Chang, J. H., and M. O. Olson. 1990. ‘Structure of the gene for rat nucleolar protein B23’, J Biol Chem, 265: 18227-33.

Chen, J., J. Sun, L. Yang, Y. Yan, W. Shi, J. Shi, Q. Huang, J. Chen, and Q. Lan. 2015. ‘Upregulation of B23 promotes tumor cell proliferation and predicts poor prognosis in glioma’, Biochem Biophys Res Commun, 466: 124-30.

Choy, E. 2012. ‘Understanding the dynamics: pathways involved in the pathogenesis of rheumatoid arthritis’, Rheumatology (Oxford), 51 Suppl 5: v3-11.

Coimbra, S., A. Figueiredo, E. Castro, P. Rocha-Pereira, and A. Santos-Silva. 2012. ‘The roles of cells and cytokines in the pathogenesis of psoriasis’, Int J Dermatol, 51: 389-95; quiz 95-8.

Consogno, G., S. Manici, V. Facchinetti, A. Bachi, J. Hammer, B. M. Conti-Fine, C. Rugarli, C. Traversari, and M. P. Protti. 2003. ‘Identification of immunodominant regions among promiscuous HLA-DR-restricted CD4+ T-cell epitopes on the tumor antigen MAGE-3’, Blood, 101: 1038-44.

Corper, A. L., T. Stratmann, V. Apostolopoulos, C. A. Scott, K. C. Garcia, A. S. Kang, I. A. Wilson, and L. Teyton. 2000. ‘A structural framework for deciphering the link between I-Ag7 and autoimmune diabetes’, Science, 288: 505-11.

Davis, M. M., J. J. Boniface, Z. Reich, D. Lyons, J. Hampl, B. Arden, and Y. Chien. 1998. ‘Ligand recognition by alpha beta T cell receptors’, Annu Rev Immunol, 16: 523-44.

Dessen, A., C. M. Lawrence, S. Cupo, D. M. Zaller, and D. C. Wiley. 1997. ‘X-ray crystal structure of HLA-DR4 (DRA*0101, DRB1*0401) complexed with a peptide from human collagen II’, Immunity, 7: 473-81.

Di Matteo, A., M. Franceschini, S. Chiarella, S. Rocchio, C. Travaglini-Allocatelli, and L. Federici. 2016. ‘Molecules that target nucleophosmin for cancer treatment: an update’, Oncotarget, 7: 44821-40.

Douat-Casassus, C., N. Marchand-Geneste, E. Diez, N. Gervois, F. Jotereau, and S. Quideau. 2007. ‘Synthetic anticancer vaccine candidates: rational design of antigenic peptide mimetics that activate tumor-specific T-cells’, J Med Chem, 50: 1598-609.

Duployez, N., L. Chebrek, N. Helevaut, E. Fournier, M. Bemba, A. Caillault, S. Geffroy, and C. Preudhomme. 2018. ‘A novel type of NPM1 mutation characterized by multiple internal tandem repeats in a case of cytogenetically normal acute myeloid leukemia’, Haematologica, 103: e575-e77.

Durrant, L. G., R. L. Metheringham, and V. A. Brentville. 2016. ‘Autophagy, citrullination and cancer’, Autophagy, 12: 1055-6.

Falini, B., C. Mecucci, E. Tiacci, M. Alcalay, R. Rosati, L. Pasqualucci, R. La Starza, D. Diverio, E. Colombo, A. Santucci, B. Bigerna, R. Pacini, A. Pucciarini, A. Liso, M. Vignetti, P. Fazi, N. Meani, V. Pettirossi, G. Saglio, F. Mandelli, F. Lo-Coco, P. G. Pelicci, M. F. Martelli, and Gimema Acute Leukemia Working Party. 2005. ‘Cytoplasmic nucleophosmin in acute myelogenous leukemia with a normal karyotype’, N Engl J Med, 352: 254-66.

Falini, B., I. Nicoletti, N. Bolli, M. P. Martelli, A. Liso, P. Gorello, F. Mandelli, C. Mecucci, and M. F. Martelli. 2007. ‘Translocations and mutations involving the nucleophosmin (NPM1) gene in lymphomas and leukemias’, Haematologica, 92: 519-32.

Feuerstein, N., P. K. Chan, and J. J. Mond. 1988. ‘Identification of numatrin, the nuclear matrix protein associated with induction of mitogenesis, as the nucleolar protein B23. Implication for the role of the nucleolus in early transduction of mitogenic signals’, J Biol Chem, 263: 10608-12.

Folch, G., D. Scaviner, V. Contet, and M. P. Lefranc. 2000. ‘Protein displays of the human T cell receptor alpha, beta, gamma and delta variable and joining regions’, Exp Clin Immunogenet, 17: 205-15.

Fremont, D. H., W. A. Hendrickson, P. Marrack, and J. Kappler. 1996. ‘Structures of an MHC class II molecule with covalently bound single peptides’, Science, 272: 1001-4.

Fremont, D. H., D. Monnaie, C. A. Nelson, W. A. Hendrickson, and E. R. Unanue. 1998. ‘Crystal structure of I-Ak in complex with a dominant epitope of lysozyme’, Immunity, 8: 305-17.

Garboczi, D. N., D. T. Hung, and D. C. Wiley. 1992. ‘HLA-A2-peptide complexes: refolding and crystallization of molecules expressed in Escherichia coli and complexed with single antigenic peptides’, Proc Natl Acad Sci USA, 89: 3429-33.

Gebauer, M., and A. Skerra. 2009. ‘Engineered protein scaffolds as next-generation antibody therapeutics’, Curr Opin Chem Biol, 13: 245-55.

Ghosh, P., M. Amaya, E. Mellins, and D. C. Wiley. 1995. ‘The structure of an intermediate in class II MHC maturation: CLIP bound to HLA-DR3’, Nature, 378: 457-62.

Godkin, A. J., K. J. Smith, A. Willis, M. V. Tejada-Simon, J. Zhang, T. Elliott, and A. V. Hill. 2001. ‘Naturally processed HLA class II peptides reveal highly conserved immunogenic flanking region sequence preferences that reflect antigen processing rather than peptide-MHC interactions’, J Immunol, 166: 6720-7.

Gonzalez-Galarza, F. F., L. Y. Takeshita, E. J. Santos, F. Kempson, M. H. Maia, A. L. da Silva, A. L. Teles e Silva, G. S. Ghattaoraya, A. Alfirevic, A. R. Jones, and D. Middleton. 2015. ‘Allele frequency net 2015 update: new features for HLA epitopes, KIR and disease and HLA adverse drug reaction associations’, Nucleic Acids Res, 43: D784-8.

Green, D. R., and B. Levine. 2014. ‘To be or not to be? How selective autophagy and cell death govern cell fate’, Cell, 157: 65-75.

Greten, T. F., and J. P. Schneck. 2002. ‘Development and use of multimeric major histocompatibility complex molecules’, Clin Diagn Lab Immunol, 9: 216-20.

Grisendi, S., C. Mecucci, B. Falini, and P. P. Pandolfi. 2006. ‘Nucleophosmin and cancer’, Nat Rev Cancer, 6: 493-505.

Grunewald, J., and A. Eklund. 2007. ‘Role of CD4+ T cells in sarcoidosis’, Proc Am Thorac Soc, 4: 461-4.

Herrera, J. E., J. J. Correia, A. E. Jones, and M. O. Olson. 1996. ‘Sedimentation analyses of the salt-and divalent metal ion-induced oligomerization of nucleolar protein B23’, Biochemistry, 35: 2668-73.

Hingorani, K., A. Szebeni, and M. O. Olson. 2000. ‘Mapping the functional domains of nucleolar protein B23’, J Biol Chem, 275: 24451-7.

Holliger, P., and G. Winter. 1993. ‘Engineering bispecific antibodies’, Curr Opin Biotechnol, 4: 446-9.

Holmberg Olausson, K., T. Elsir, K. Moazemi Goudarzi, M. Nister, and M. S. Lindstrom. 2015. ‘NPM1 histone chaperone is upregulated in glioblastoma to promote cell survival and maintain nucleolar shape’, Sci Rep, 5: 16495.

Holmdahl, R., L. Klareskog, K. Rubin, E. Larsson, and H. Wigzell. 1985. ‘T lymphocytes in collagen II-induced arthritis in mice. Characterization of arthritogenic collagen II-specific T-cell lines and clones’, Scand J Immunol, 22: 295-306.

Hoppes, R., R. Oostvogels, J. J. Luimstra, K. Wals, M. Toebes, L. Bies, R. Ekkebus, P. Rijal, P. H. Celie, J. H. Huang, M. E. Emmelot, R. M. Spaapen, H. Lokhorst, T. N. Schumacher, T. Mutis, B. Rodenko, and H. Ovaa. 2014. ‘Altered peptide ligands revisited: vaccine design through chemically modified HLA-A2-restricted T cell epitopes’, J Immunol, 193: 4803-13.

Hosken, N. A., and M. J. Bevan. 1990. ‘Defective presentation of endogenous antigen by a cell line expressing class I molecules’, Science, 248: 367-70.

Huston, J. S., D. Levinson, M. Mudgett-Hunter, M. S. Tai, J. Novotny, M. N. Margolies, R. J. Ridge, R. E. Bruccoleri, E. Haber, R. Crea, and et al. 1988. ‘Protein engineering of antibody binding sites: recovery of specific activity in an anti-digoxin single-chain Fv analogue produced in Escherichia coli’, Proc Natl Acad Sci USA, 85: 5879-83.

Ireland, J. M., and E. R. Unanue. 2011. ‘Autophagy in antigen-presenting cells results in presentation of citrullinated peptides to CD4 T cells’, J Exp Med, 208: 2625-32.

Jairajpuri, Z. S., S. Rana, S. Khetrapal, M. A. Talikoti, and S. Jetley. 2014. ‘Extranodal anaplastic large cell lymphoma mimicking sarcoma: A report of an interesting case’, IntJ Appl Basic Med Res, 4: S50-2.

Jian, Y., Z. Gao, J. Sun, Q. Shen, F. Feng, Y. Jing, and C. Yang. 2009. ‘RNA aptamers interfering with nucleophosmin oligomerization induce apoptosis of cancer cells’, Oncogene, 28: 4201-11.

June, C. H., M. V. Maus, G. Plesa, L. A. Johnson, Y. Zhao, B. L. Levine, S. A. Grupp, and D. L. Porter. 2014. ‘Engineered T cells for cancer therapy’, Cancer Immunol Immunother, 63: 969-75.

Jurtz, V., S. Paul, M. Andreatta, P. Marcatili, B. Peters, and M. Nielsen. 2017. ‘NetMHCpan-4.0: Improved Peptide-MHC Class I Interaction Predictions Integrating Eluted Ligand and Peptide Binding Affinity Data’, J Immunol, 199: 3360-68.

Kang, Y. J., M. O. Olson, and H. Busch. 1974. ‘Phosphorylation of acid-soluble proteins in isolated nucleoli of Novikoff hepatoma ascites cells. Effects of divalent cations’, J Biol Chem, 249: 5580-5.

Kang, Y. J., M. O. Olson, C. Jones, and H. Busch. 1975. ‘Nucleolar phosphoproteins of normal rat liver and Novikoff hepatoma ascites cells’, Cancer Res, 35: 1470-5.

Karlin, S., and S. F. Altschul. 1993. ‘Applications and statistics for multiple high-scoring segments in molecular sequences’, Proc Natl Acad Sci USA, 90: 5873-7.

Kim, A., I. Z. Hartman, B. Poore, T. Boronina, R. N. Cole, N. Song, M. T. Ciudad, R. R. Caspi, D. Jaraquemada, and S. Sadegh-Nasseri. 2014. ‘Divergent paths for the selection of immunodominant epitopes from distinct antigenic sources’, Nat Commun, 5: 5369.

Kovats, S., P. E. Whiteley, P. Concannon, A. Y. Rudensky, and J. S. Blum. 1997. ‘Presentation of abundant endogenous class II DR-restricted antigens by DM-negative B cell lines’, Eur J Immunol, 27: 1014-21.

Kuo, Y. H., Y. T. Chen, H. P. Tsai, C. Y. Chai, and A. L. Kwan. 2015. ‘Nucleophosmin overexpression is associated with poor survival in astrocytoma’, APMIS, 123: 515-22.

Latek, R. R., A. Suri, S. J. Petzold, C. A. Nelson, O. Kanagawa, E. R. Unanue, and D. H. Fremont. 2000. ‘Structural basis of peptide binding and presentation by the type I diabetes-associated MHC class II molecule of NOD mice’, Immunity, 12: 699-710.

Lee, K. H., K. W. Wucherpfennig, and D. C. Wiley. 2001. ‘Structure of a human insulin peptide-HLA-DQ8 complex and susceptibility to type 1 diabetes’, Nat Immunol, 2: 501-7.

Lee, S. B., T. L. Xuan Nguyen, J. W. Choi, K. H. Lee, S. W. Cho, Z. Liu, K. Ye, S. S. Bae, and J. Y. Ahn. 2008. ‘Nuclear Akt interacts with B23/NPM and protects it from proteolytic cleavage, enhancing cell survival’, Proc Natl Acad Sci U S A, 105: 16584-9.

Lefranc, M. P. 2001. ‘Nomenclature of the human T cell receptor genes’, Curr Protoc Immunol, Appendix 1: Appendix 10. ---. 2003. ‘IMGT databases, web resources and tools for immunoglobulin and T cell receptor sequence analysis, http://imgt.cines.fr’, Leukemia, 17: 260-6.

Leotoing, L., L. Meunier, M. Manin, C. Mauduit, M. Decaussin, G. Verrijdt, F. Claessens, M. Benahmed, G. Veyssiere, L. Morel, and C. Beaudoin. 2008. ‘Influence of nucleophosmin/B23 on DNA binding and transcriptional activity of the androgen receptor in prostate cancer cell’, Oncogene, 27: 2858-67.

Li, Y., H. Li, R. Martin, and R. A. Mariuzza. 2000. ‘Structural basis for the binding of an immunodominant peptide from myelin basic protein in different registers by two HLA-DR2 proteins’, J Mol Biol, 304: 177-88.

Li, Y., R. Moysey, P. E. Molloy, A. L. Vuidepot, T. Mahon, E. Baston, S. Dunn, N. Liddy, J. Jacob, B. K. Jakobsen, and J. M. Boulter. 2005. ‘Directed evolution of human T-cell receptors with picomolar affinities by phage display’, Nat Biotechnol, 23: 349-54.

Li, Y. P., R. K. Busch, B. C. Valdez, and H. Busch. 1996. ‘C23 interacts with B23, a putative nucleolar-localization-signal-binding protein’, EurJ Biochem, 237: 153-8.

Liddy, N., G. Bossi, K. J. Adams, A. Lissina, T. M. Mahon, N. J. Hassan, J. Gavarret, F. C. Bianchi, N. J. Pumphrey, K. Ladell, E. Gostick, A. K. Sewell, N. M. Lissin, N. E. Harwood, P. E. Molloy, Y. Li, B. J. Cameron, M. Sami, E. E. Baston, P. T. Todorov, S. J. Paston, R. E. Dennis, J. V. Harper, S. M. Dunn, R. Ashfield, A. Johnson, Y. McGrath, G. Plesa, C. H. June, M. Kalos, D. A. Price, A. Vuidepot, D. D. Williams, D. H. Sutton, and B. K. Jakobsen. 2012. ‘Monoclonal TCR-redirected tumor cell killing’, Nat Med, 18: 980-7.

Lindstrom, M. S. 2011. ‘NPM⅟B23: A Multifunctional Chaperone in Ribosome Biogenesis and Chromatin Remodeling’, Biochem Res Int, 2011: 195209.

Liu, Q. R., and P. K. Chan. 1993. ‘Characterization of seven processed pseudogenes of nucleophosmin/B23 in the human genome’, DNA Cell Biol, 12: 149-56.

Liu, S., J. Matsuzaki, L. Wei, T. Tsuji, S. Battaglia, Q. Hu, E. Cortes, L. Wong, L. Yan, M. Long, A. Miliotto, N. W. Bateman, S. B. Lele, T. Chodon, R. C. Koya, S. Yao, Q. Zhu, T. P. Conrads, J. Wang, G. L. Maxwell, A. A. Lugade, and K. Odunsi. 2019. ‘Efficient identification of neoantigen-specific T-cell responses in advanced human ovarian cancer’, J Immunother Cancer, 7: 156.

Lo-Coco, F., G. Avvisati, M. Vignetti, C. Thiede, S. M. Orlando, S. lacobelli, F. Ferrara, P. Fazi, L. Cicconi, E. Di Bona, G. Specchia, S. Sica, M. Divona, A. Levis, W. Fiedler, E. Cerqui, M. Breccia, G. Fioritoni, H. R. Salih, M. Cazzola, L. Melillo, A. M. Carella, C. H. Brandts, E. Morra, M. von Lilienfeld-Toal, B. Hertenstein, M. Wattad, M. Lubbert, M. Hanel, N. Schmitz, H. Link, M. G. Kropp, A. Rambaldi, G. La Nasa, M. Luppi, F. Ciceri, O. Finizio, A. Venditti, F. Fabbiano, K. Dohner, M. Sauer, A. Ganser, S. Amadori, F. Mandelli, H. Dohner, G. Ehninger, R. F. Schlenk, U. Platzbecker, dell’Adulto Gruppo Italiano Malattie Ematologiche, Group German-Austrian Acute Myeloid Leukemia Study, and Leukemia Study Alliance. 2013. ‘Retinoic acid and arsenic trioxide for acute promyelocytic leukemia’, N Engl J Med, 369: 111-21.

Martelli, M. P., I. Gionfriddo, F. Mezzasoma, F. Milano, S. Pierangeli, F. Mulas, R. Pacini, A. Tabarrini, V. Pettirossi, R. Rossi, C. Vetro, L. Brunetti, P. Sportoletti, E. Tiacci, F. Di Raimondo, and B. Falini. 2015. ‘Arsenic trioxide and all-trans retinoic acid target NPM1 mutant oncoprotein

levels and induce apoptosis in NPM1-mutated AML cells’, Blood, 125: 3455-65. McCormack, E., K. J. Adams, N. J. Hassan, A. Kotian, N. M. Lissin, M. Sami, M. Mujic, T. Osdal, B. T. Gjertsen, D. Baker, A. S. Powlesland, M. Aleksic, A. Vuidepot, O. Morteau, D. H. Sutton, C. H. June, M. Kalos, R. Ashfield, and B. K. Jakobsen. 2013. ‘Bi-specific TCR-anti CD3 redirected T-cell targeting of NY-ESO-1- and LAGE-1-positive tumors’, Cancer Immunol Immunother, 62: 773-85.

Mendes-da-Silva, P., A. Moreira, J. Duro-da-Costa, D. Matias, and C. Monteiro. 2000. ‘Frequent loss of heterozygosity on chromosome 5 in non-small cell lung carcinoma’, Mol Pathol, 53: 184-7.

Metheringham, R. L., V. A. Pudney, B. Gunn, M. Towey, I. Spendlove, and L. G. Durrant. 2009. ‘Antibodies designed as effective cancer vaccines’, MAbs, 1: 71-85.

Morris, S. W., M. N. Kirstein, M. B. Valentine, K. G. Dittmer, D. N. Shapiro, D. L. Saltman, and A. T. Look. 1994. ‘Fusion of a kinase gene, ALK, to a nucleolar protein gene, NPM, in non-Hodgkin’s lymphoma’, Science, 263: 1281-4.

Munz, C. 2012. ‘Antigen Processing for MHC Class II Presentation via Autophagy’, Front Immunol, 3: 9.

Myers, E. W., and W. Miller. 1989. ‘Approximate matching of regular expressions’, Bull Math Biol, 51: 5-37.

Namboodiri, V. M., I. V. Akey, M. S. Schmidt-Zachmann, J. F. Head, and C. W. Akey. 2004. ‘The structure and function of Xenopus NO38-core, a histone chaperone in the nucleolus’, Structure, 12: 2149-60.

Nozawa, Y., N. Van Belzen, A. C. Van der Made, W. N. Dinjens, and F. T. Bosman. 1996. ‘Expression of nucleophosmin/B23 in normal and neoplastic colorectal mucosa’, J Pathol, 178: 48-52.

Nygren, P. A. 2008. ‘Alternative binding proteins: affibody binding proteins developed from a small three-helix bundle scaffold’, FEBSJ, 275: 2668-76.

Okuwaki, M., K. Matsumoto, M. Tsujimoto, and K. Nagata. 2001. ‘Function of nucleophosmin/B23, a nucleolar acidic protein, as a histone chaperone’, FEBS Lett, 506: 272-6.

Pearson, W. R., and D. J. Lipman. 1988. ‘Improved tools for biological sequence comparison’, Proc Natl Acad Sci USA, 85: 2444-8.

Pianta, A., C. Puppin, A. Franzoni, D. Fabbro, C. Di Loreto, S. Bulotta, M. Deganuto, I. Paron, G. Tell, E. Puxeddu, S. Filetti, D. Russo, and G. Damante. 2010. ‘Nucleophosmin is overexpressed in thyroid tumors’, Biochem Biophys Res Commun, 397: 499-504.

Pluckthun, A. 1991. ‘Antibody engineering: advances from the use of Escherichia coli expression systems’, Biotechnology (N Y), 9: 545-51.

Qi, W., K. Shakalya, A. Stejskal, A. Goldman, S. Beeck, L. Cooke, and D. Mahadevan. 2008. ‘NSC348884, a nucleophosmin inhibitor disrupts oligomer formation and induces apoptosis in human cancer cells’, Oncogene, 27: 4210-20.

Redner, R. L., E. A. Rush, S. Faas, W. A. Rudert, and S. J. Corey. 1996. ‘The t(5;17) variant of acute promyelocytic leukemia expresses a nucleophosmin-retinoic acid receptor fusion’, Blood, 87: 882-6.

Reff, M. E. 1993. ‘High-level production of recombinant immunoglobulins in mammalian cells’, Curr Opin Biotechnol, 4: 573-6.

Remington, RP. 1980. Remington’s pharmaceutical sciences (Mack Pub. Co).

Robins, H. S., P. V. Campregher, S. K. Srivastava, A. Wacher, C. J. Turtle, O. Kahsai, S. R. Riddell, E. H. Warren, and C. S. Carlson. 2009. ‘Comprehensive assessment of T-cell receptor beta-chain diversity in alphabeta T cells’, Blood, 114: 4099-107.

Sambrook, J, Fritsch and Maniatis. 1989. Molecular cloning: A laboratory manual (Cold Spring Harbor Laboratory Press).

Sato, K., R. Hayami, W. Wu, T. Nishikawa, H. Nishikawa, Y. Okuda, H. Ogata, M. Fukuda, and T. Ohta. 2004. ‘Nucleophosmin/B23 is a candidate substrate for the BRCA1-BARD1 ubiquitin ligase’, J Biol Chem, 279: 30919-22.

Schmid, D., M. Pypaert, and C. Munz. 2007. ‘Antigen-loading compartments for major histocompatibility complex class II molecules continuously receive input from autophagosomes’, Immunity, 26: 79-92.

Scott, C. A., P. A. Peterson, L. Teyton, and I. A. Wilson. 1998. ‘Crystal structures of two I-Ad-peptide complexes reveal that high affinity can be achieved without large anchor residues’, Immunity, 8: 319-29.

Sekhar, K. R., M. Benamar, A. Venkateswaran, S. Sasi, N. R. Penthala, P. A. Crooks, S. R. Hann, L. Geng, R. Balusu, T. Abbas, and M. L. Freeman. 2014. ‘Targeting nucleophosmin 1 represents a rational strategy for radiation sensitization’, IntJ Radiat Oncol Biol Phys, 89: 1106-14.

Sette, A., L. Adorini, S. M. Colon, S. Buus, and H. M. Grey. 1989. ‘Capacity of intact proteins to bind to MHC class II molecules’, J Immunol, 143: 1265-7.

Shields, L. B., C. Gercel-Taylor, C. M. Yashar, T. C. Wan, W. A. Katsanis, J. A. Spinnato, and D. D. Taylor. 1997. ‘Induction of immune responses to ovarian tumor antigens by multiparity’, J Soc Gynecol Investig, 4: 298-304.

Skaar, T. C., S. C. Prasad, S. Sharareh, M. E. Lippman, N. Brunner, and R. Clarke. 1998. ‘Two-dimensional gel electrophoresis analyses identify nucleophosmin as an estrogen regulated protein associated with acquired estrogen-independence in human breast cancer cells’, J Steroid Biochem Mol Biol, 67: 391-402.

Skerra, A. 2008. ‘Alternative binding proteins: anticalins - harnessing the structural plasticity of the lipocalin ligand pocket to engineer novel binding activities’, FEBSJ, 275: 2677-83.

Smith, K. J., J. Pyrdol, L. Gauthier, D. C. Wiley, and K. W. Wucherpfennig. 1998. ‘Crystal structure of HLA-DR2 (DRA*0101, DRB1*1501) complexed with a peptide from human myelin basic protein’, J Exp Med, 188: 1511-20.

Stern, L. J., J. H. Brown, T. S. Jardetzky, J. C. Gorga, R. G. Urban, J. L. Strominger, and D. C. Wiley. 1994. ‘Crystal structure of the human class II MHC protein HLA-DR1 complexed with an influenza virus peptide’, Nature, 368: 215-21.

Stewart. 1984. Solid phase peptide synthesis (Illinois Pierce Chemical Company: Rockford).

Subong, E. N., M. J. Shue, J. I. Epstein, J. V. Briggman, P. K. Chan, and A. W. Partin. 1999. ‘Monoclonal antibody to prostate cancer nuclear matrix protein (PRO:4-216) recognizes nucleophosmin/B23’, Prostate, 39: 298-304.

Swaminathan, V., A. H. Kishore, K. K. Febitha, and T. K. Kundu. 2005. ‘Human histone chaperone nucleophosmin enhances acetylation-dependent chromatin transcription’, Mol Cell Biol, 25: 7534-45.

Szebeni, A., J. E. Herrera, and M. O. Olson. 1995. ‘Interaction of nucleolar protein B23 with peptides related to nuclear localization signals’, Biochemistry, 34: 8037-42.

Szebeni, A., and M. O. Olson. 1999. ‘Nucleolar protein B23 has molecular chaperone activities’, Protein Sci, 8: 905-12.

Tanaka, M., H. Sasaki, I. Kino, T. Sugimura, and M. Terada. 1992. ‘Genes preferentially expressed in embryo stomach are predominantly expressed in gastric cancer’, Cancer Res, 52: 3372-7.

Tanikawa, C., K. Ueda, H. Nakagawa, N. Yoshida, Y. Nakamura, and K. Matsuda. 2009. ‘Regulation of protein Citrullination through p53/PADI4 network in DNA damage response’, Cancer Res, 69: 8761-9.

Thomsen, M. C., and M. Nielsen. 2012. ‘Seq2Logo: a method for construction and visualization of amino acid binding motifs and sequence profiles including sequence weighting, pseudo counts and two-sided representation of amino acid enrichment and depletion’, Nucleic Acids Res, 40: W281-7.

Torelli, A., and C. A. Robotti. 1994. ‘ADVANCE and ADAM: two algorithms for the analysis of global similarity between homologous informational sequences’, Comput Appl Biosci, 10: 3-5.

Traunecker, A., A. Lanzavecchia, and K. Karjalainen. 1991. ‘Bispecific single chain molecules (Janusins) target cytotoxic lymphocytes on HIV infected cells’, EMBOJ, 10: 3655-9.

Trill, J. J., A. R. Shatzman, and S. Ganguly. 1995. ‘Production of monoclonal antibodies in COS and CHO cells’, Curr Opin Biotechnol, 6: 553-60.

Tsui, K. H., A. J. Cheng, Pe Chang, T. L. Pan, and B. Y. Yung. 2004. ‘Association of nucleophosmin/B23 mRNA expression with clinical outcome in patients with bladder carcinoma’, Urology, 64: 839-44.

Wang, D., H. Umekawa, and M. O. Olson. 1993. ‘Expression and subcellular locations of two forms of nucleolar protein B23 in rat tissues and cells’, Cell Mol Biol Res, 39: 33-42.

Ward, E. S., D. Gussow, A. D. Griffiths, P. T. Jones, and G. Winter. 1989. ‘Binding activities of a repertoire of single immunoglobulin variable domains secreted from Escherichia coli’, Nature, 341: 544-6.

Watt, P. M. 2006. ‘Screening for peptide drugs from the natural repertoire of biodiverse protein folds’, Nat Biotechnol, 24: 177-83.

Wulff, J. E., R. Siegrist, and A. G. Myers. 2007. ‘The natural product avrainvillamide binds to the oncoprotein nucleophosmin’, JAm Chem Soc, 129: 14444-51.

Xue, W., R. L. Metheringham, V. A. Brentville, B. Gunn, P. Symonds, H. Yagita, J. M. Ramage, and L. G. Durrant. 2016. ‘SCIB2, an antibody DNA vaccine encoding NY-ESO-1 epitopes, induces potent antitumor immunity which is further enhanced by checkpoint blockade’, Oncoimmunology, 5: e1169353.

Yoneda-Kato, N., A. T. Look, M. N. Kirstein, M. B. Valentine, S. C. Raimondi, K. J. Cohen, A. J. Carroll, and S. W. Morris. 1996. ‘The t(3;5)(q25.1;q34) of myelodysplastic syndrome and acute myeloid leukemia produces a novel fusion gene, NPM-MLF1’, Oncogene, 12: 265-75.

Yun, J. P., E. C. Chew, C. T. Liew, J. Y. Chan, M. L. Jin, M. X. Ding, Y. H. Fai, H. K. Li, X. M. Liang, and Q. L. Wu. 2003. ‘Nucleophosmin/B23 is a proliferate shuttle protein associated with nuclear matrix’, J Cell Biochem, 90: 1140-8.

Yun, J. P., J. Miao, G. G. Chen, Q. H. Tian, C. Q. Zhang, J. Xiang, J. Fu, and P. B. Lai. 2007. ‘Increased expression of nucleophosmin/B23 in hepatocellular carcinoma and correlation with clinicopathological parameters’, Br J Cancer, 96: 477-84.

Zhu, X., H. J. Belmont, S. Price-Schiavi, B. Liu, H. I. Lee, M. Fernandez, R. L. Wong, J. Builes, P. R. Rhode, and H. C. Wong. 2006. ‘Visualization of p53(264-272)/HLA-A*0201 complexes naturally presented on tumor cell surface by a multimeric soluble single-chain T cell receptor’, J Immunol, 176: 3223-32.

Zhu, Y., M. Shi, H. Chen, J. Gu, J. Zhang, B. Shen, X. Deng, J. Xie, X. Zhan, and C. Peng. 2015. ‘NPM1 activates metabolic changes by inhibiting FBP1 while promoting the tumorigenicity of pancreatic cancer cells’, Oncotarget, 6: 21443-51.

1. A citrullinated T cell antigen which comprises, consists essentially of, or consists of
i) the amino acid sequence AKFINYVKNCFRMTD, wherein the arginine (R) residue is replaced with citrulline, or
ii) the amino acid sequence of i), with the exception of 1, 2 or 3 amino acid substitutions, and/or 1, 2 or 3 amino acid insertions, and/or 1, 2 or 3 amino acid deletions in a non-arginine position.

2. The antigen of claim 1, which comprises, consists essentially of, or consists of
i) one or more of the following amino acid sequences:
AKFINYVKNCFRMTDQEAIQ
LPKVEAKFINYVKNCFRMTD
wherein the arginine (R) residue is replaced with citrulline, or
ii) one or more of the amino acid sequences of i), with the exception of 1, 2 or 3 amino acid substitutions, and/or 1, 2 or 3 amino acid insertions, and/or 1, 2 or 3 amino acid deletions in a non-citrulline position.

3. A complex of the antigen of claim 1 or claim 2 and an MHC molecule, optionally wherein the MHC molecule is MHC class II, optionally selected from HLA-DR4 and DP4.

4. A binding moiety that binds the polypeptide of claim 1 or claim 2.

5. The binding moiety of claim 4, which binds the polypeptide when it is in complex with MHC.

6. The binding moiety of claim 4 or claim 5, wherein the binding moiety is a T cell receptor (TCR) or an antibody.

7. The binding moiety of claim 6, wherein the TCR is on the surface of a cell.

8. An antigen as defined in claim 1 or claim 2, a complex as defined in claim 3, or a binding moiety as defined in any one of claims 4-7 for use in medicine.

9. The antigen, complex, and/or binding moiety for use as defined in claim 8 for use in treating or preventing cancer.

10. The antigen, complex, and/or binding moiety for use as defined in claim 9, wherein the cancer is AML, lung, colorectal, renal, breast, ovary and liver tumours.

11. A pharmaceutical composition comprising an antigen as defined in claim 1 or claim 2, a complex as defined in claim 3, and/or a binding moiety as defined in any one of claims 4-7, together with a pharmaceutically acceptable carrier.

12. A method of identifying a binding moiety that binds a complex as claimed in claim 3, the method comprising contacting a candidate binding moiety with the complex and determining whether the candidate binding moiety binds the complex.
